SEQUENCE LISTING (1 sequence)

SEQ ID NO: 1 - DNA, 52 bp, Homo Sapiens
  misc_feature (27)...(27): N is either A or T
gtctggccgacagtactgcctggctgngtctaactggaggagagtctaactg

(19) United States
(12) Patent Application Publication
(10) Pub. No.: US 2009/0203012 A1
(43) Pub. Date: Aug. 13, 2009

(54) Metabolic Syndrome Genetics

(75) Inventors: Vincent Mooser, King of Prussia, PA (US); Dawn Waterworth, King of Prussia, PA (US); KiJoung Song, King of Prussia, PA (US); Xin Yuan, King of Prussia, PA (US); Christopher W. Knouff, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: SMITHKLINE BEECHAM CORPORATION, Philadelphia, PA (US)

(21) Appl. No.: 12/336,769

(22) Filed: Dec. 17, 2008

Related U.S. Application Data
(60) Provisional application No. 61/018,939, filed on Jan. 4, 2008.

(51) Int. Cl. C12Q 1/68; C12Q 1/48

(52) U.S. Cl. 435/6; 435/15

(57) ABSTRACT

A gene (and polymorphisms within it) was associated with multiple components of the metabolic syndrome. Methods of screening subjects to identify risk of metabolic syndrome and related conditions are described, as are methods of screening compounds to identify those that act on the gene product as a target for the treatment of these conditions.

### FIELD OF THE INVENTION

The present invention relates to the identification of the association of a gene (and polymorphisms within it) with multiple components of the metabolic syndrome; to methods of screening subjects to identify risk of metabolic syndrome and related conditions, including dyslipidemia, obesity, type 2 diabetes, hypertension, non-alcoholic fatty liver, coronary artery disease; and to screening methods to identify compounds that act on the gene product as a target for the treatment of these conditions.

### BACKGROUND OF THE INVENTION

The metabolic syndrome is a cluster of cardiovascular and metabolic risk factors, with an as-yet undefined underlying pathology. Adverse clinical sequelae associated with metabolic syndrome are cardiovascular disease (including coronary heart disease) and Type 2 diabetes mellitus (T2D). Recognized components of metabolic syndrome include abdominal obesity, atherogenic dyslipidemia, increased blood pressure, and insulin resistance. The metabolic syndrome has also been linked with prothrombotic and proinflammatory states. See e.g., Grundy Circulation. 109:433-438 (2004).

People diagnosed with the metabolic syndrome are at increased risk of coronary heart disease, other diseases related to plaque buildups in artery walls (e.g., stroke and peripheral vascular disease), and type 2 diabetes. Two longitudinal studies in men without cardiovascular disease at baseline showed that the metabolic syndrome was associated with an adverse cardiovascular outcome. Isomaa et al. Diabetes Care 24:683-689 (2001). Lakka et al. JAMA 288:2709-2716 (2002). Marroquin et al. (Circulation 109:714-721, 2004) showed that women with the metabolic syndrome had a lower 4-year survival than women without metabolic syndrome. In addition, evidence from cross-sectional studies indicate that the risk for Alzheimer disease is also increased in presence of metabolic syndrome. In one particular study, Vanhanen et al. observed a OR=2.5 (1.3-4.8) of having Alzheimer disease in elderly individuals with metabolic syndrome [Neurology 67:843-7 (2006)]

Currently, first-line therapy for individuals with metabolic syndrome is primarily directed at lifestyle changes such as dietary changes and weight reduction. Drug treatment to reduce insulin resistance may also be used, as well as other therapies targeted to the individual components of metabolic syndrome.

Identification of common physiologic pathways underlying the multiple components of the metabolic syndrome will be useful in developing improved therapeutic approaches to metabolic syndrome. The present investigators elucidated genetic markers and genes that are associated with metabolic syndrome, leading to methods of screening subjects to identify risk of metabolic syndrome, and to screening methods to identify compounds that act on the associated gene product and that may be developed as medicines to treat metabolic syndrome and/or its associated pathologies.

### SUMMARY OF THE INVENTION

An object of the present invention is a method of identifying a subject's relative risk of developing metabolic syndrome, comprising determining the allelic genotype at the STK39/SPAK rs16855027 single nucleotide polymorphism loci. An T/T genotype indicates the subject has a reduced risk of developing metabolic syndrome compared to an individual with an A/T or A/A genotype.

A further object is a method of identifying a subject's relative risk of developing a condition selected from hypertension, Type 2 diabetes mellitus (T2D), cardiovascular disease, and dyslipidemia, the method comprising determining the allelic genotype at the STK39/SPAK rs16855027 single nucleotide polymorphism loci. An T/T genotype indicates said subject has a reduced risk of developing said condition compared to an individual with an A/T or A/A genotype.

A further object is a method of identifying, in a population of overweight subjects, subpopulations at different relative risk of developing a condition selected from hypertension, T2D, and cardiovascular disease, the method comprising determining the allelic genotype at the STK39/SPAK rs16855027 single nucleotide polymorphism loci for each subject in the population. The subpopulation having an T/T genotype is at reduced risk of developing said condition compared to the subpopulation with the A/T and A/A genotypes.

A further object is a method of screening a test compound for use as a therapeutic for metabolic syndrome, comprising determining whether said compound inhibits the kinase activity of STK39/SPAK. Inhibitory activity indicates potential as a treatment for metabolic syndrome or the associated components or sequelae of metabolic syndrome.

A further object of the present invention is a method of treating a condition selected from metabolic syndrome, hypertension, cardiovascular disease and T2D, comprising administering a therapeutic compound that inhibits the kinase activity of STK39/SPAK.

### BRIEF DESCRIPTION OF THE FIGURES

FIG. 1 shows the genomic context of STK39/SPAK, and the association found in the present study between multiple STK39/SPAK SNPs and both systolic and diastolic blood pressure. Numbers at the top of the Figure refer to the position of each SNP on chromosome2. In the graphs, the y-axis is the degree of association (expressed as −log 10 p value), and the vertical bars show the association of each SNP with systolic blood pressure (upper graph) and diastolic blood pressure (lower graph).

FIG. 2A shows the mean value for Z-score transformed log values for components of metabolic syndrome related to BMI (i.e. BMI, waist measurement, hip measurement, height, % fat, adiponectin, leptin; see also Table 1), for the three STK39/SPAK rs16855027 SNP genotypes (A/A, A/T, T/T). Levels of significance are depicted in Table 1.

FIG. 2B shows, as a way of comparison, the mean value for Z-score transformed log values for components of the metabolic syndrome related to BMI (i.e. BMI, waist measurement, hip measurement, height, % fat, adiponectin, leptin) as in FIG. 2A, for the three FTO rs9939609 SNP genotypes (T/T, A/T and A/A).

FIG. 3A shows the mean value for Z-score transformed log values for components of metabolic syndrome related to blood pressure, lipids, and inflammation (i.e., SBP, DBP, HDL, LDL, TG, ALT, CRP), for the three STK39/SPAK rs16855027 SNP genotypes (A/A, A/T, T/T).

FIG. 3B shows the mean value for Z-score transformed log values for components of metabolic syndrome related to blood pressure, lipids, and inflammation as in FIG. 3A, for the three FTO rs9939609_SNP genotypes (T/T, A/T and A/A).

FIG. 4A shows the mean value for Z-score transformed log values for components of metabolic syndrome related to glucose metabolism (i.e., glucose, insulin, HOMA, Albuminuria) for the three STK39/SPAK rs16855027 SNP genotypes (A/A, A/T, T/T).

FIG. 4B shows the mean value for Z-score transformed log values for components of metabolic syndrome related to glucose metabolism as in FIG. 4A, for the three FTO rs9939609_SNP genotypes (T/T, A/T and A/A).

FIG. 5 compares the differences in Z-score transformed variables between homozygous carriers of the rare and common alleles for FTO rs9939609 SNP (squares), and the rare (T) and common (A) alleles for STK39/SPAK rs16855027 SNP (circles).

FIG. 6 shows the interactors of STK39/SPAK Of note is the presence of numerous cation transporters, as well as the With No Lysine (K) kinases WNK-1 and -4, and proteins involved in signalling.

FIG. 7A shows the prevalence of metabolic syndrome according to STK39/SPAK rs16855027 SNP genotype (A/A, A/T, T/T) in 5,446 subjects.

FIG. 7B shows the prevalence of T2D according to STK39/SPAK rs16855027 SNP genotype (A/A, A/T, T/T) in the same 5,446 subjects as in FIG. 7A.

FIG. 8 is a flow chart summary of the sampling procedure used in the CoLaus study.

FIG. 9A indicates that, when using an unmatched case-control study design, the CoLaus study is able to detect genotype/phenotype associations depending on the number of cases and estimated effect size. FIG. 9a shows an arbitrary allelic frequency of 0.3, a disease prevalence of 50% and a type 1 error rate of 10⁻⁷ taking into account 500,000 genetic markers. Curves are for estimated genetic effects (odds ratios) of 1.2 to 1.8. In the CoLaus study, for hypertension with 2268 cases, the estimated power is >0.9 for a 1.4 effect size, and >0.5 for a 1.3 effect size.

FIG. 9B shows power calculations using a continuous outcome for independent subjects, with an additive mode of action for the allele and a type 1 error rate of 10⁻⁷. Calculations were done for various minor allele frequencies (0.1 to 0.5). SBP is an example of continuous trait analysis in the CoLaus study. For allelic frequencies of 0.2 to 0.4, the study has an estimated power of >0.8 to detect BP variations of 2.0-2.3 mm Hg.

### DETAILED DESCRIPTION OF THE INVENTION

### BMI and Obesity

In the United States, the National Institutes of Health classifies body weight using the concept of body mass index (National Institutes of Health, National Heart, Lung and Blood Institute. Clinical guidelines on the identification, evaluation, and treatment of overweight and obesity in adults. Bethesda, Md.: U.S. Department of Health and Human Services, 1998). Body Mass Index (BMI) is calculated by dividing the body weight in kilograms by the square of the height in meters. Overweight is typically defined as a BMI above 25 kg/m² but less than 30 kg/m², and obesity is defined as a BMI of 30 kg/m² or greater.

While obesity is associated with chronic complications, including cardiovascular disease, not every obese subject will experience such complications. The distribution of adipose tissue has been associated with cardiovascular risk. A higher waist circumference identifies increased metabolic risk (measuring waist circumference takes into account the distribution of adipose tissue). For example, for the same level of BMI, the cardiovasular risk may be significantly lower in a person whose excess body fat is predominantly in a gluteal-femoral distribution versus an abdominal distribution. (See, eg., Haffner et al., Obesity, 14 Suppl 3:121S-127S (2006); Despres, J. Endocrinol. Invest. 29(3 Suppl):77-82 (2006)).

### Metabolic Syndrome

The metabolic syndrome (also referred to as ‘syndrome X’ or Primary Insulin Resistance Syndrome) is a cluster of cardiovascular and metabolic risk factors, with an underlying pathology thought to be linked to insulin resistance. The main clinical outcomes associated with metabolic syndrome are coronary heart disease and Type 2 diabetes mellitus (T2D). Recognized components of metabolic syndrome include abdominal obesity, atherogenic dyslipidemia, increased blood pressure, and insulin resistance. The metabolic syndrome has also been linked with prothrombotic and proinflammatory states. See e.g., Grundy Circulation. 109:433-438 (2004).

Atherogenic dyslipidemia includes raised triglycerides and low concentrations of HDL cholesterol, as well as other lipoprotein abnormalities such as increased remnant lipoproteins, elevated apolipoprotein B, small LDL particles, and small HDL particles.

A proinflammatory state can be recognized clinically by elevated C-reactive protein (CRP). A prothrombotic state is characterized by increased plasma plasminogen activator inhibitor (PAI)-1 and fibrinogen.

Commonly used definitions of the metabolic syndrome are those proposed by the World Health Organization (WHO) (Alberti et al., Diabet Med 15:539-553 (1998); Einhorn et al., Endocrin Pract 9:237-252 (2003)) and the Third Report of the National Cholesterol Education Program Expert Panel on Detection, Evaluation, and Treatment of High Blood Cholesterol in Adults (Adult Treatment Panel III [ATP III]), Circulation 106:3143-3421 (2002). The criteria for metabolic syndrome include waist circumference, serum triglycerides, blood HDL-cholesterol level, blood pressure, and serum glucose.

The risk factors of metabolic syndrome may be regulated by both genetic and acquired factors, which leads to variability in the occurrence of risk factors. For example, genetic variation may modulate lipoprotein metabolism, and thus dyslipidemias associated with obesity and/or insulin resistance vary considerably.

### Current Studies

The present inventors performed a detailed analysis of the STK39/SPAK gene in the extensively phenotyped CoLaus population-based Study from Lausanne, Switzerland, and identified a series of genetic variants (SNPs) associated with blood pressure levels. Additionally, it was identified that homozygous carriers of the minor allele (T/T) for the Single Nucleotide Polymorphism (SNP) rs16855027 had a marked reduction in multiple components of metabolic syndrome: blood pressure, BMI, waist and hip circumference, body fat mass, triglycerides levels, insulin resistance and C-Reactive Protein (CRP). Type 2 diabetes and hypertension were also not as prevalent in homozygous (T/T) rs16855027 carriers. This represents the first genetic variant found to be associated with multiple major components of metabolic syndrome. As such, these findings establish STK39/SPAK as a potential target for the prevention and/or treatment of metabolic syndrome, as well as the symptoms and components of the metabolic syndrome and related conditions.

### The Lausanne Study

The CoLaus population-based study from Lausanne Switzerland was designed to understand the phenotypic and molecular genetic architecture of cardiovascular and common psychiatric disorders in a Caucasian population (Appendix 1: The CoLaus study: a population-based study to investigate the molecular architecture of cardiovascular risk factors and metabolic syndrome; Firmann et al). Briefly, it comprises 6205 extensively phenotyped individuals aged 35-75 years, randomly selected from the general population (see Appendix 1; also Mathieu Firmann, et al., The CoLaus study: a population-based sample for the study of the epidemiology and molecular architecture of cardiovascular risk factors. Submitted for publication.). Out of these 6205 participants, 6000 participants have been genome-wide genotyped using the Affymetrix 500K Single Nucleotide Polymorphism (SNP) chip. The Lausanne study has been used in studies of the frequency of common cardiovascular and metabolic risk factors in a European population (Vollenweider et al., Rev Med Suisse 2006; 2(86):2528-3), and for pharmaco-epidemiology purposes (Marques-Vidal et al., Eur J Clin Nutr 2007 [Epub ahead of print]; Rodondi et al., Prev Med 2007. Aug. 23; [Epub ahead of print]).

The present inventors used the Lausanne study to investigate the biology of the STK39/SPAK gene in humans.

### STK39/SPAK

Human STK39 (serine threonine kinase 39; NP 037365) is also known as SPAK (STE20/SPS1-related, proline alanine rich kinase). STK39/SPAK is a kinase which appears to be involved in the phosphorylation of Na+-K+-2CL-co-transporter by WNK1 [With No Lysine (K) 1] and WNK4, and to be involved in sodium reabsorption in the distal nephron. See, e.g., Yan et al., Biochim Biophys Acta. 2007 February; 1769(2):106-16. Activation of WNK1 coincides with the phosphorylation and activation of STK39/SPAK and oxidative stress response kinase-1 (OSR1), both substrates for WNK1. Zagorska et al. report that small interfering RNA depletion of WNK1 impairs STK39/OSR1 activity and the phosphorylation of residues targeted by WNK1. (Zagorska et al. J. Cell Biol. 2007 Jan. 1; 176(1):89-100.)

Intronic deletions in WNK1 are reported to be associated with the autosomal dominant Gordon's hypertension syndrome. (See, e.g., O'Reilly et al., J Am Soc Nephrol. 2003 October; 14(10):2447-56.)

Anselmo et al. (PNAS 103(29): 10883-8, July 2006) suggest that WNK1 regulates OSR1, STK39/SPAK, and NKCC activities, and that OSR1 and STK39/SPAK are links between WNK1 and NKCC in a pathway that contributes to volume regulation and blood pressure homeostasis in mammals.

Physical and physiological processes of STK39/SPAK are reviewed in Delpire et al., SPAK and OSR1, key kinases involved in the regulation of chloride transport. Acta Physiol (Oxf). 2006 May-June; 187(1-2):103-13. Biochemical characterization of STK39/SPAK catalytic activity demonstrates that it is a serine/threonine kinase that can phosphorylate itself and an exogenous substrate in vitro. Full-length STK39/SPAK is reported to be expressed in the cytoplasm in transfected cells.

Johnston et al. state that the similarity of STK39/SPAK to other SPS1 family members, its ability to activate the p38 pathway, in addition to its putative caspase cleavage site, provide evidence that STK39/SPAK may act as a mediator of stress-activated signals [Oncogene (2000) 19, 4290-4297]. Piechotta et al. also suggest that STK39/SPAK is an intermediate in the cell's response to cellular stress. J Biol Chem. 2003 Dec. 26; 278(52):52848-56; and Piechotta et al., J Biol Chem. 2002 Dec. 27; 277(52):50812-9. Tsutsumi et al. report that STK39/SPAK may be involved in the regulation of the cytoskeleton in response to cellular stresses such as hyperosmotic shock. J Biol Chem. 2000 Mar. 31; 275(13):9157-62.

An association between STK39/SPAK polymorphism and hypertension in three populations, including two Amish populations, was reported in October 2007 at the annual meeting of the American Society for Human Genetics (Wang et al., Whole-genome association study in the Old Order Amish identifies STK39/SPAK as a novel hypertension susceptibility gene; Presented at the Annual Meeting of the American Society for Human Genetics, 25 Oct. 2007, San Diego, Calif. Available from www.ashg.org.)

### Diagnostic Screening Methods

Methods of identifying subjects with metabolic syndrome (or at increased risk of developing metabolic syndrome) at an early stage will be useful in providing the proper advice on non-pharmacological interventions (i.e. dietary and physical activity) and medical treatment to such subjects. It is recognized that not all obese or over-weight individuals will develop the constellation of symptoms known as metabolic syndrome; a method of identifying whether an overweight person is at increased risk or reduced risk of metabolic syndrome will be useful in providing individualized medical treatment.

The present inventors have established that a correlation exists between an individual's STK39/SPAK genotype, and the risk of developing metabolic syndrome. Accordingly, a method of assessing an individual's relative risk of developing metabolic syndrome involves genotyping that individual at the SNK39 gene to determine whether the individual's genotype places them at increased risk of metabolic syndrome. Individuals with a genotype that has been associated with an increased incidence of metabolic syndrome (compared to the incidence of metabolic syndrome in subjects with alternate genotypes) are at increased risk of developing metabolic syndrome.

As used herein, “genotyping” a subject (or a DNA sample) for polymorphic allele(s) of a gene(s) means detecting which allelic or polymorphic form(s) of the gene(s) is present in a subject (or a sample). As is known in the art, an individual may be heterozygous or homozygous for a particular allele. More than two allelic forms may exist, thus there may be more than three possible genotypes. For purposes of the present invention, “genotyping” includes the determination of STK39/SPAK alleles using any suitable techniques, as are known in the art. As used herein, an allele may be ‘detected’ when other possible allelic variants have been ruled out.

As used herein, a “genetic subset” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., all individuals within a certain geography, a group of individuals having a certain phenotype (e.g., hypertension, obesity), individuals diagnosed with a specific condition, individuals of a particular ethnic background. It is known that the frequency of a particular allele may differ among populations of different ethnic backgrounds.

As used herein, “an individual of Caucasian descent” refers to people who have ancestors from the geographic region of Northern, Eastern, or Central Europe. Generally the individuals have light skin color and are from regions including, but not limited to, Western Europe, North America, England, Russia, and Germany.

As used herein, a subject that is “predisposed to” or “at increased risk of” a particular phenotype, or of developing a particular condition, based on genotyping will be more likely to display that phenotype or condition than an individual with a different genotype at the target polymorphic locus (or loci).

“Genetic testing” (also called genetic screening) as used herein refers to the testing of a biological sample from a subject to determine the subject's genotype; and may be utilized to determine if the subject's genotype comprises alleles that are either associated with, cause, or increase susceptibility to, a particular phenotype or condition.

Therefore, in one embodiment, the invention provides a method of assessing an individual's predisposition to metabolic syndrome and its accompanying complications such as T2D, and/or cardiovascular disease including coronary heart disease. The method comprises identifying and analyzing the STK39/SPAK polymorphisms in an isolated nucleic acid sample taken from the individual, wherein presence of STK39/SPAK rs16855027 genotype T/T in a subject indicates a reduced risk of metabolic syndrome, compared to the risk of metabolic syndrome in a population of subjects with alternate STK39/SPAK rs16855027 genotypes (A/T, A/A). In another embodiment, the presence of STK39/SPAK rs16855027 genotype T/T in a subject indicates a reduced risk of developing a condition selected from T2D, hypertension, dyslipidemia, and cardiovascular disease, compared to the risk of the same condition in a population of subjects with alternate STK39/SPAK rs16855027 genotypes (A/T, A/A). Thus in one embodiment, the present genotyping methods are used prognostically, to determine an overweight or obese individual's relative risk of developing metabolic syndrome.

In one embodiment, the individual tested is of Caucasian descent and the reduced risk is as compared to a population of Caucasians with alternate STK39/SPAK rs16855027 genotypes (A/T, A/A).

Another embodiment of the present methods is a method of dividing a starting population into genetic subpopulations that are at different (increased and decreased) risk of having or developing a conditions selected from metabolic syndrome, T2D, hypertension, dyslipidemia, and cardiovascular disease. In one embodiment, a starting population of subjects is genotyped at the STK39/SPAK rs16855027 allele, and divided into a genetic subpopulation of those at increased risk of such conditions (A/T or A/A genotypes) and those with reduced risk (T/T genotype). The starting population may consist of overweight or obese subjects, or subjects of a single ethnicity (such as Caucasian). Such methods may be useful in selecting subjects for appropriate medical treatment and/or in designing clinical trials.

Biological samples used in the present methods include solid materials (e.g., tissue, cell pellets, biopsies) and biological fluids (e.g. blood, saliva). Nucleic acid molecules of the instant invention include DNA and RNA and can be isolated from a particular biological sample using any of a number of procedures which are known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russell, Cold Spring Harbor Laboratory Press, 2001.

### Drug Compound Screening Methods

A further embodiment of the present invention is a method of screening test compounds to identify those with activity that may be useful in treating or preventing metabolic syndrome, or the components or sequelae of metabolic syndrome, in a mammalian subject (including humans). The method comprises screening a test compound for the ability to inhibit the kinase activity of STK39/SPAK, where inhibitory activity indicates potential use as a treatment for metabolic syndrome or the associated components or sequelae of metabolic syndrome, or use as a method of preventing cardiovascular disease, hypertension, dyslipidemia, or, T2D in subjects at increased risk of developing metabolic syndrome.

A further embodiment of the present invention is a method of treating metabolic syndrome or its components or sequelae, by administering to a mammal in need of treatment a compound that inhibits the kinase activity of STK39/SPAK. The compound is administered as a pharmaceutical composition comprising an STK39/SPAK inhibitor and a pharmaceutically acceptable carrier.

### EXAMPLES

### Example 1

### Methods

The design of the Lausanne study is described in Appendix 1.

The association between STK39/SPAK gene variants and binary traits or continuous variables was assessed by logistic and linear regression analysis, respectively, using additive or recessive models. Degree of significance was determined after adjustment for age, gender, geographical location and BMI, where indicated. Geographical components were used to take into account population stratification and were computed using EIGENSOFT (http://genepath.med.harvard.edu/˜reich/Software.htm); the first ten geographical components were used in these analyses. Analyses were carried out using PLINK version 0.99 p (http://pngu.mgh.harvard.edu/purcell/plink/; Purcell et al., Am J Hum Genet, 81(3):559 (2007)). A p-value≦0.05 was considered significant. In addition, association between rs16855027 and continuous variables was examined after Z-score transformation of raw data, or log-transformed data for non-normally distributed variables, using mean and SD values computed from the whole collection. Outliers, i.e. Z-score values ≦−3 or ≧+3 units were removed from this analysis. Genetic associations were visualized in their genomic context using the genome browser on the human assemblies (NCBI Build 36). Known protein coding genes were taken from the NCBI mRNA reference sequences collection.

### Example 2

### Associations Between STK39/SPAK SNPs and Blood Pressure

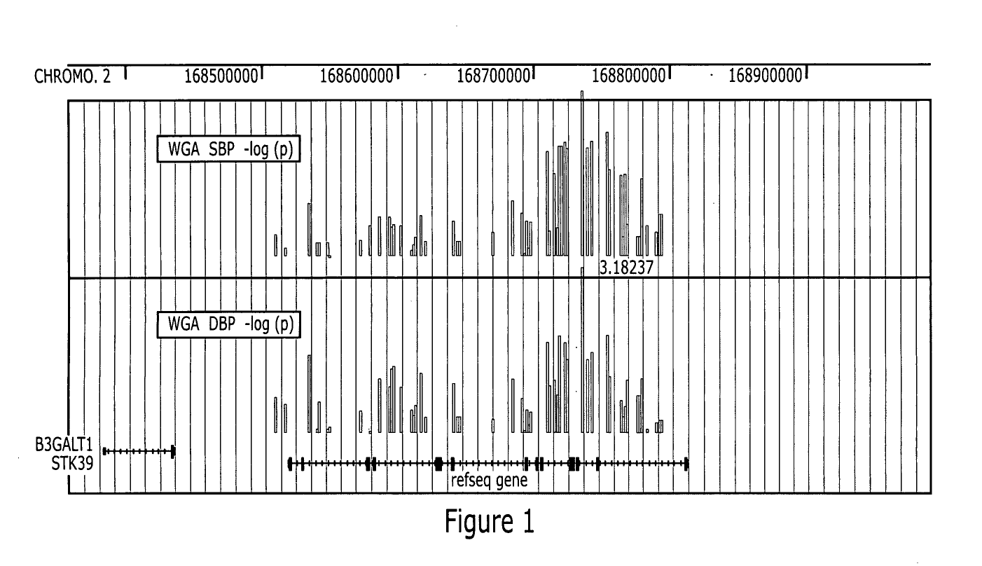

Associations between Single Nucleotide Polymorphisms (SNPs) within the STK39/SPAK locus and systolic and diastolic blood pressure were examined using both a recessive and an additive model. The recessive model provided a better fit for the association. Overall, the recessive analysis demonstrated that multiple SNPs within one haplotype block of the STK39/SPAK gene were strongly associated with blood pressure in the Lausanne population. FIG. 1 shows the genomic context of STK39/SPAK, and the association found in the present study between multiple STK39/SPAK SNPs and both systolic and diastolic blood pressure. Numbers at the top of the Figure refer to the position of each SNP on chromosome2. In the graphs, the y-axis is the degree of association (expressed as −log 10 p value), and the vertical bars show the association of each SNP with systolic blood pressure (upper graph) and diastolic blood pressure (lower graph).

Each of the STK39/SPAK hypertension-associated SNPs identified were intronic, with no variants found that coded for non-synonymous amino acid changes. The following STK39/SPAK SNPs were tested for association with SBP, with the p value shown for the recessive model:

| SNP | P value |
|---|---|
| RS9789702 | 0.3409 |
| RS4668002 | 0.2718 |
| RS10180407 | 0.8054 |
| RS16854601 | 0.6629 |
| RS4667551 | 0.4489 |
| RS6433032 | 0.1777 |
| RS12479070 | 0.5538 |
| RS16854635 | |
| RS4667553 | 0.896 |
| RS4233815 | 0.2127 |
| RS16854676 | 0.5504 |
| RS12692873 | 0.3613 |
| RS1816977 | 0.08924 |
| RS4668021 | 0.2854 |
| RS1356374 | 0.1573 |
| RS2138753 | 0.9817 |
| RS12618884 | 0.2221 |
| RS13419175 | 0.7532 |
| RS10175836 | |
| RS3769412 | 0.2342 |
| RS1517329 | 0.009813 |
| RS1850438 | 0.3383 |
| RS1400641 | 0.02691 |
| RS10170500 | 0.3039 |
| RS6714609 | 0.02642 |
| RS6714707 | 0.0293 |
| RS2063958 | 0.007734 |
| RS3769393 | 0.006614 |
| RS3769392 | 0.008213 |
| RS3754776 | |
| RS3769390 | |
| RS7589259 | 0.01958 |
| RS10497337 | 0.003869 |
| RS16855027 | 0.000655 |
| RS6740492 | 0.01892 |
| RS6740826 | 0.2656 |
| RS4668040 | |
| RS11890527 | 0.008168 |
| RS11896742 | 0.009087 |
| RS10177198 | |
| RS10192362 | 0.2445 |
| RS-GSK48213056 | 0.5302 |
| RS2203703 | 0.006132 |
| RS1878486 | |
| RS11898447 | |
| RS16855079 | 0.004295 |
| RS13385577 | 0.02163 |
| RS10202854 | 0.02259 |
| RS4667570 | 0.023 |
| RS16855092 | |
| RS4668046 | 0.02884 |
| RS17797694 | 0.4712 |
| RS6728947 | 0.9335 |
| RS7605161 | 0.02739 |
| RS16855116 | 0.2539 |
| RS16855133 | 0.4663 |
| RS4438452 | 0.0335 |
| RS4613238 | 0.5427 |
| RS10208207 | 0.2747 |
| RS-GSK48214017 | |
| RS2030162 | 0.3658 |
| RS1356373 | 0.4926 |
| RS1829227 | |
| RS10497338 | |
| RS1017666 | 0.1679 |
| RS16855155 | |

The SNP showing the strongest association with both systolic and diastolic blood pressure was rs16855027, which is an A/T SNP:

GTCTGGCCGACAGTACTGCCTGGCTG[A/T]GTCTAACTGGAGGAGAGTC
TAACTG

This SNP corresponds to the tallest bars on FIG. 1.

### Example 3

### STK39/SPAK SNP rs16855027 and Components of the Metabolic Syndrome

The association between the rs16855027 SNP and various phenotypic traits related to Metabolic Syndrome and collected in the CoLaus Study is depicted in Table 1 below. As can be seen in Table 1, the rs16855027 SNP is associated not only with blood pressure levels, but with other components of the metabolic syndrome including lipids (triglycerides), adiposity (body weight, fat content, waist and hip circumference) and inflammation (CRP). The association is in a recessive manner, with homozygous carriers of the rare allele (T/T) being protected from these conditions. Overall, the effect sizes of this SNP are large: the rare homozygotes (T/T) show a 5 mmHg lower systolic and 3 mmHg lower diastolic blood pressure than either heterozygotes (A/T) or common (A/A) homozygotes. Compared to A/A and A/T carriers, T/T homozygotes also showed a 3 cm lower waist circumference, a 21% lower HOMA, a 14% lower triglyceride and a 19% lower CRP levels in plasma. Overall, this analysis indicates that homozygous carriers of the STK39/SPAK minor T allele at SNP rs16855027 are markedly protected against multiple facets of metabolic syndrome. Furthermore, these associations between STK39/SPAK SNP16855027 remained significant for certain variables after adjustment for BMI, indicating that the effects of this SNP on metabolic syndrome-related traits is only partly mediated by the effect on obesity.

TABLE 1
Clinical characteristics of the CoLaus Study participants
according to STK39/SPAK rs16855027 SNP genotype.

| | Genotype N (%) | | | | |
|---|---|---|---|---|---|
| | TT 140 | AT 1564 | AA 3742 | | |
| Variable | (2.6%) | (28.7%) | (68.7%) | P Additive | P Recessive |
| Gender (% F) | 52.9 | 53.7 | 52.6 | 0.5599 | 0.9019 |
| Age (years) | 52.6 ± 10.8 | 52.9 ± 10.7 | 52.1 ± 10.5 | 0.0213 | 0.7773 |
| Weight (kg) | 69.8 ± 12.4 | 72.6 ± 12.8 | 72.5 ± 12.8 | 0.2866 | 0.0097 |
| Height (cm) | 168.9 ± 6.5 | 168.5 ± 6.5 | 168.6 ± 6.5 | 0.8479 | 0.5651 |
| BMI (kg/m2) | 24.5 ± 4.1 | 25.6 ± 4.3 | 25.5 ± 4.3 | 0.2997 | 0.0027 |
| Waist (cm) | 85.6 ± 11.1 | 88.9 ± 11.5 | 88.7 ± 11.4 | 0.2428 | 0.0009 |
| Hip (cm) | 99.8 ± 9.0 | 101.5 ± 9.0 | 101.4 ± 9 | 0.4514 | 0.0356 |
| Fat content (%) | 18.3 ± 7.0 | 20.3 ± 7.5 | 20.2 ± 7.5 | 0.1688 | 0.0014 |
| Body fat (kg) | 26.2 ± 6.5 | 27.9 ± 6.8 | 27.8 ± 6.8 | 0.2048 | 0.0030 |
| Fat free mass (kg) | 50.3 ± 7.6 | 50.9 ± 7.6 | 50.9 ± 7.6 | 0.8821 | 0.3868 |
| Leptin (ng/mL) | 7 ± 6.6 | 8.6 ± 7.9 | 8.5 ± 7.9 | 0.2599 | 0.0105 |
| Adiponectin (mg/ml) | 8.4 ± 6.3 | 8 ± 6.0 | 8.2 ± 6.1 | 0.5423 | 0.6479 |
| Glucose (mmol/L) | 5.4 ± 0.9 | 5.5 ± 0.9 | 5.5 ± 0.9 | 0.7801 | 0.5561 |
| Insulin (mIU/mL) | 5.1 ± 4.6 | 6.2 ± 5.7 | 6.1 ± 5.6 | 0.3199 | 0.0117 |
| *HOMA | 1.2 ± 1.2 | 1.6 ± 1.5 | 1.5 ± 1.5 | 0.2985 | 0.0028 |
| T2Diabetes (% total) | 4 (21.1) | 117 (40.9) | 241 (36.6) | 0.9000 | 0.0334 |
| Micro-albuminuria (mg/ml) | 7.3 ± 8.5 | 8.4 ± 9.1 | 8.4 ± 9 | 0.4789 | 0.1347 |
| Total cholesterol (mmol/L) | 5.4 ± 1.0 | 5.5 ± 1.1 | 5.5 ± 1.1 | 0.9629 | 0.1532 |
| LDL-cholesterol (mmol/L) | 3.3 ± 1.0 | 3.4 ± 1.0 | 3.3 ± 1 | 0.6100 | 0.2851 |
| HDL-cholesterol (mmol/L) | 1.63 ± 0.4 | 1.56 ± 0.4 | 1.58 ± 0.4 | 0.5015 | 0.0986 |
| Triglycerides (mmol/L) | 1 ± 0.6 | 1.2 ± 0.6 | 1.2 ± 0.6 | 0.5880 | 0.0009 |
| SBP (mm Hg) | 125.4 ± 17.2 | 130 ± 17.6 | 130.7 ± 17.7 | 0.0049 | 0.0007 |
| Diastolic BP (mm Hg) | 77.1 ± 11.4 | 80.4 ± 11.8 | 80.5 ± 11.8 | 0.0421 | 0.0007 |
| Hypertension (% total) | 23 (30.3) | 349 (47.3) | 846 (45.9) | 0.3730 | 0.0072 |
| Heart rate (beats/min) | 66.8 ± 10.0 | 67.2 ± 9.9 | 67.3 ± 9.9 | 0.5127 | 0.5944 |
| GFR (mL/min) | 78.6 ± 15.4 | 77.2 ± 14.9 | 76.8 ± 14.8 | 0.1666 | 0.1986 |
| GGT (IU/L) | 22.6 ± 15.2 | 24.2 ± 15.6 | 24.2 ± 15.5 | 0.5673 | 0.2162 |
| ALT (IU/L) | 23.1 ± 11.3 | 24.6 ± 11.6 | 24.3 ± 11.4 | 0.8713 | 0.2011 |
| AST (IU/L) | 27.5 ± 8.9 | 28.1 ± 8.8 | 28.1 ± 8.8 | 0.6182 | 0.4602 |
| Albumin (g/L) | 43.9 ± 2.6 | 44.1 ± 2.6 | 44.1 ± 2.6 | 0.331 | 0.2037 |
| C-reactive protein (mg/dL) | 1.07 ± 1.3 | 1.33 ± 1.5 | 1.34 ± 1.5 | 0.1621 | 0.0216 |
| Calcium (mmol/L) | 2.3 ± 0.1 | 2.3 ± 0.1 | 2.3 ± 0.1 | 0.3457 | 0.3116 |
| Protein (g/L) | 73.7 ± 4.5 | 74.3 ± 4.5 | 74.3 ± 4.5 | 0.5482 | 0.1271 |
| Homocystein (mmol/L) | 9.5 ± 2.9 | 9.9 ± 3.0 | 9.9 ± 3 | 0.8576 | 0.1142 |
| **Metabolic syndrome(%) | 11 (7.9) | 226 (14.5) | 537 (14.4) | 0.1496 | 0.0184 |

Data are expressed as mean ± SD, or percent.
*HOMA = homeostatic Model Assessment of insulin resistance, see e.g., Wallace et al., Diabetes Care 27 (6): 1487-95. (2004)
**Metabolic syndrome defined using the NCEP-ATP III criteria.

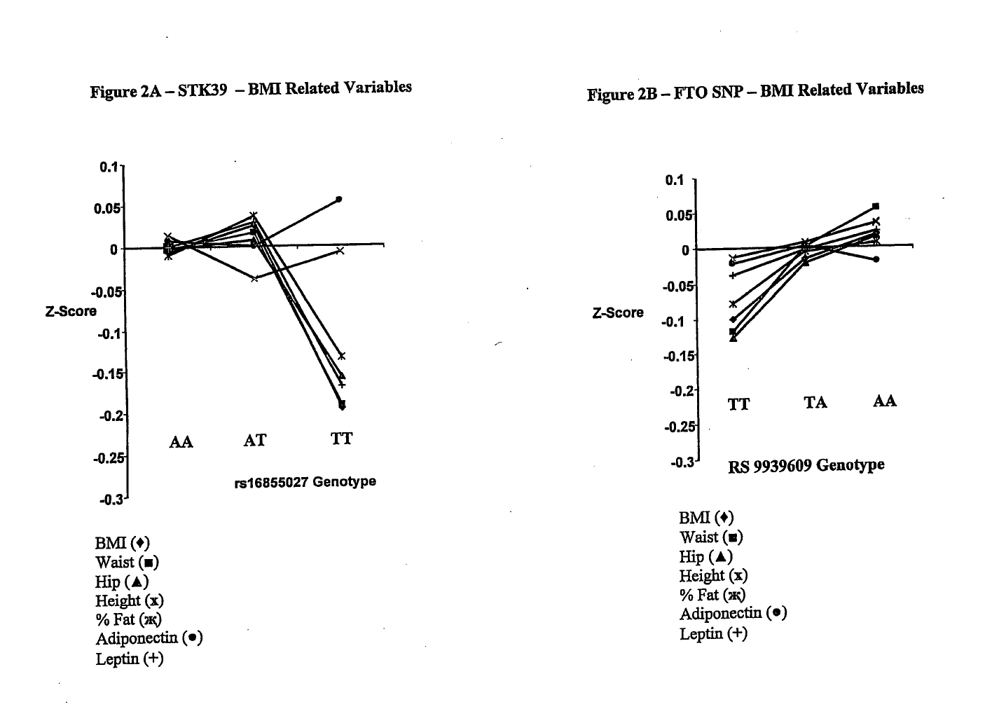

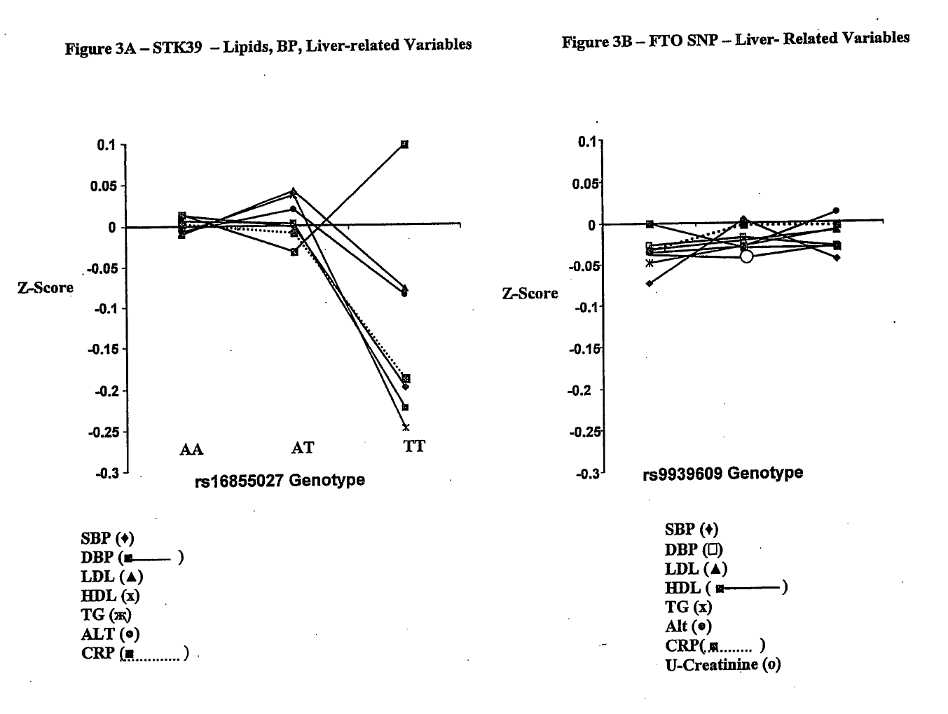

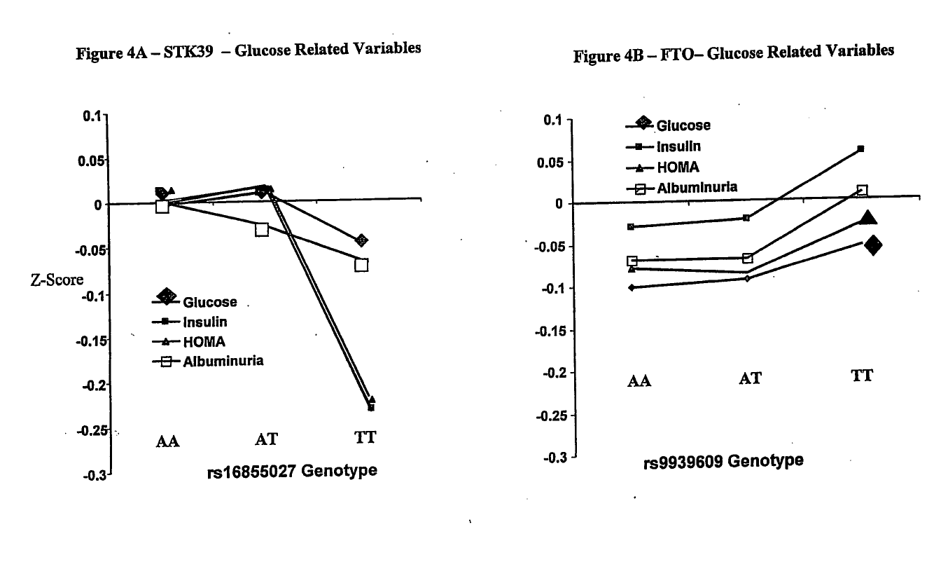

To further characterize the association between the STK39/SPAK rs16855027 SNP and metabolic syndrome components, and to show them on the same Figure, all variables were transformed into Z-score values, corresponding to the number of SD units departing from the mean value for the entire group. FIGS. 2A, 3A and 4A illustrate the mean value for Z-score transformed log values, according to the three STK39/SPAK rs16855027 genotypes (A/A, A/T, T/T). A recessive mode of transmission, as well as the effect of the homozygous T/T genotype on components of metabolic syndrome, i.e. body weight-related (FIG. 2A), blood pressure-, lipid- and inflammation-related (FIG. 3A) and glucose-related (FIG. 4A) variables, is clearly apparent.

To test the hypothesis that the effect on blood pressure is mediated by obesity, the associations were recalculated after adjusting for waist circumference. Systolic and diastolic blood pressures remained significantly associated with STK39/SPAK gene, though the significance was attenuated (p=0.007 and 0.01, respectively). Triglycerides also remained significantly association (p=0.02). These results suggest that the STK39/SPAK gene may have independent effects on obesity, blood pressure, and lipids.

### Example 3

### Demonstration that Distinct Obesity-Associated Genes have Different Metabolic Effects

The FTO gene (see Peters et al, Cloning of Fatso (Fto), a novel gene deleted by the Fused toes (Ft) mouse mutation”, Mamm. Genome 10 (10): 983-6 1999) has recently been associated with obesity (see e.g., Frayling et al, Science 316(5826):889 (2007); Dina et al Nat Genet 39(6):724-6. 2007; Scuteri et al., PLoS Genet 3(7):e115 (2007)); Scott et al., Science 316(5829):1341-5. 2007; Saxena et al., Science 316(5829):1331-6 2007).

A comparison of the effect of STK39/SPAK rs16855027 with the effect of FTO rs9939609 SNP is illustrated in FIGS. 2-4 (STK39/SPAK shown in FIGS. 2A, 3A and 4A; FTO shown in FIGS. 2B, 3B and 4B). This comparison indicates that the rare allele for FTO predisposes to obesity in a dominant manner and to diabetes in a most likely recessive manner, whereas the rare allele for STK39/SPAK protects against multiple components of the metabolic syndrome in a recessive manner.

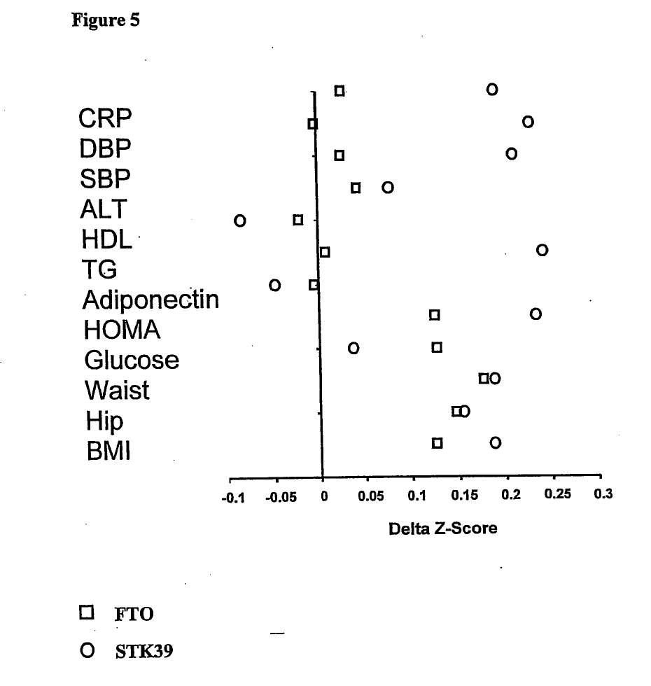

FIG. 5 further illustrates the different effects of STK39/SPAK and FTO on metabolic traits. Squares on FIG. 5 indicate the difference (the delta) between Z-score transformed variables for homozygous carriers of the rare FTO allele and for homozygous carriers of the common FTO allele; circles indicate the delta Z-score of variables for homozygous carriers of the common STK39/SPAK allele and for homozygous carriers of the rare STK39/SPAK allele. For example, the DBP delta Z-score for FTO is about 0, indicating that there was no difference in DBP measurements between FTO rs9939609 AA and TT genotypes.

As indicated on FIG. 5, for waist and hip measurements there is a relatively similar effect for STK39/SPAK rs16855027 homozygosity and FTO rs9939609 homozygosity, i.e., for both STK39/SPAK and FTO there was a similar delta Z-score for waist and hip when comparing the homozygous genotypes (see also FIGS. 2A and 2B). However, as shown on FIG. 5, the effect of homozygous genotype on other variables differed between the two genes; see e.g.:
CRP: delta Z-score of <0.05 for FTO; delta Z-score of >0.15 for STK39/SPAK,
DBP: delta Z-score of about 0 for FTO; delta Z-score of >0.2 for STK39/SPAK,
SBP: delta Z-score of <0.05 for FTO; delta Z-score of >0.2 for STK39/SPAK; and
Triglycerides: delta Z-score of <0.05 for FTO; delta Z-score of >0.2 for STK39/SPAK.
   These differences between STK39/SPAK and FTO were statistically significant. These data provide the first demonstration for a genetic predisposition explaining the metabolic heterogeneity among obese individuals.

### Example 4

### Further Analysis of the STK39/SPAK Gene

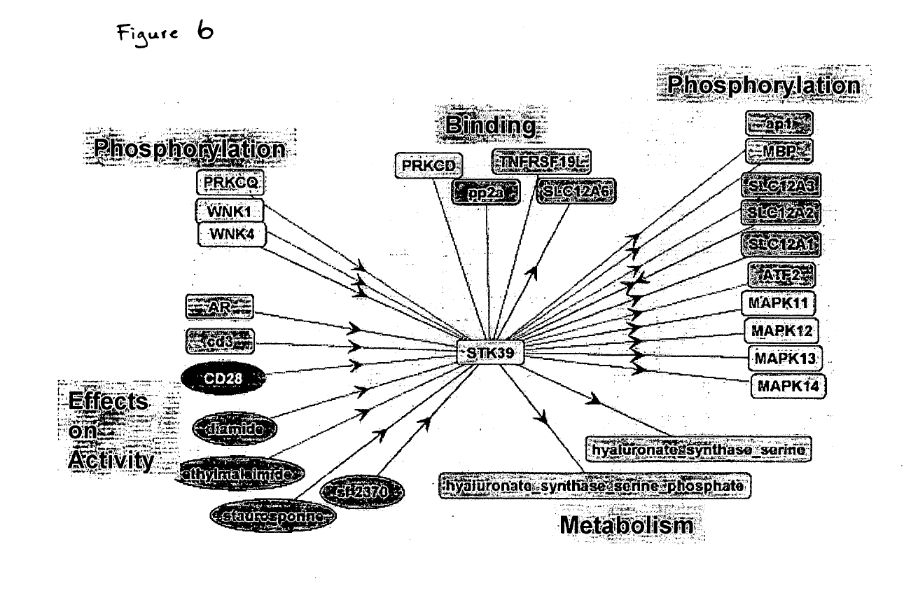

To gain additional insight into the biology of STK39/SPAK, a NETUNO analysis was done. The interactors of STK39/SPAK are shown in FIG. 6. Of note is the presence of numerous cation transporters, as well as the With No Lysine (K) kinases WNK-1 and -4, and proteins involved in signalling STK39/SPAK activates by phosphorylation a number of related transporters (SLC12A1/2/3). SLC12A1 is kidney specific and is known to be sensitive to loop diuretics such as furosemide and butamide. SLC12A3 is also kidney specific and is known to be thiazide sensitive. SLC12A2 is expressed in the intestines and bronchus.

The tissue distribution of STK39/SPAK gene expression was examined using the GeneLogic database (which measures RNA expression, results not shown). The STK39/SPAK gene was found to be relatively ubiquitously expressed, with a particularly high expression in the brain, moderate expression in the gut and kidney, and low expression in the liver (data not shown).

### Example 5

### Prevalence of Metabolic Syndrome and T2D

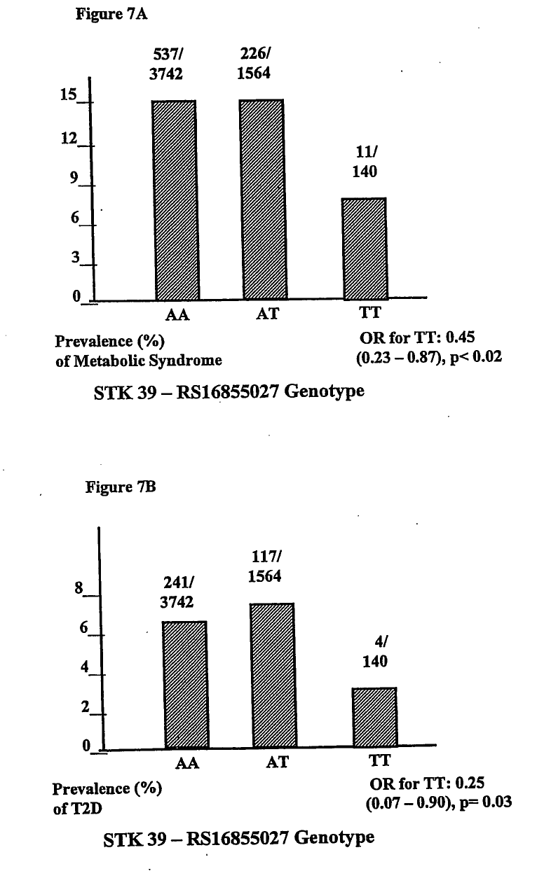

FIGS. 7A and 7B show the prevalence of Metabolic syndrome and T2D in 5,446 subjects genotyped for STK39/SPAK rs16855027. Shown on the x-axis are the 3 genotypes for this particular SNP, whereas the y-axis denotes the relative proportion (in %) of the carriers of each genotype who meet the NCEP-ATP-III definition for metabolic syndrome. Absolute numbers are shown on top of each column. The odds ratio (OR) of having metabolic syndrome in homozygous carriers of the rare allele (T/T) is 0.45 (95% confidence interval 0.23-0.87) compared to the other genotypes, corresponding to a 55% (95% CJ 13%-77%) reduction in risk.

### APPENDIX 1

### The CoLaus Study: a Population-Based Study to Investigate the Molecular Architecture of Cardiovascular Risk Factors and Metabolic Syndrome

Mathieu Firmann^{1#} M.D., Vladimir Mayor^{1#} M.D., Pedro Marques Vidal² M.D., Murielle Bochud² M.D.Ph.D., Alain Pécoud³ M.D., Daniel Hayoz⁴ M.D., Fred Paccaud² M.D., Martin Preisig⁵ M.D., Kijoung S. Song⁶ Ph.D., Xin Yuan⁶ Ph.D., Theodore M. Danoff M.D.⁷, Heide A Stimadel⁸ Ph.D., Dawn Waterworth⁶ Ph.D., Vincent Mooser6 M.D., Gérard Waeber¹ M.D. and Peter Vollenweider¹ M.D. 1. Department of Medicine, Internal Medicine, CHUV, Lausanne, Switzerland2. Institute of Social and Preventive Medicine (IUMSP), University of Lausanne, Switzerland3. Outpatient Clinic, University of Lausanne, Switzerland4. Department of Medicine, Angiology, CHUV, Lausanne, Switzerland5. Department of Psychiatry, CHUV, Lausanne, Switzerland6. Medical Genetics/Clinical Pharmacology and Discovery Medicine 7. Center of Excellence for Drug Discovery CV and 8. Worldwide Epidemiology, GlaxoSmithKline, Philadelphia, Pa., USA# These authors contributed equally to this work

### Address for Correspondence and Reprints

### Peter Vollenweider, MD

Service de Médecine Interne

### Centre Hospitalier Universitaire Vaudois

### Rue du Bugnon 46

### 1011 Lausanne

### Abstract

Objective: To present the rationale, design, methods and first results of the CoLaus study, a project aimed at assessing the prevalence and deciphering the molecular determinants of cardiovascular risk factors (CVRFs) in the Caucasian population of Lausanne, Switzerland.
Methods: Single-center, cross-sectional study including a random sample of 6,188 extensively phenotyped Caucasian subjects (3,251 women and 2,937 men) aged 35 to 75 years living in Lausanne, and genotyped using the 500K Affymetrix chip technology.
Results: The prevalence of obesity, smoking, hypertension, dyslipidemia and diabetes were 15.7%, 27.0%, 36.7%, 34.2% and 6.6%, respectively, and was greater in men than in women.
In both genders, the prevalence of obesity, hypertension and diabetes increased with age. Initial genetic analyses confirmed the presence of a significant association between selected candidate-genes, used as positive controls, and selected CV-related conditions.
Conclusion: Given its size, the depth of the phenotypic analysis, the elevated prevalence of CV risk factors (CVRFs) and the availability of dense genome-wide genetic data, the CoLaus Study is a unique resource to investigate the epidemiology of isolated, or aggregated CVRFs like the metabolic syndrome, and can serve as a discovery set, as well as replication set, to identify novel genes associated with these conditions.
Keywords: genome-wide association study, epidemiology, cardiovascular risk factors.

### Introduction

Cardiovascular diseases (CVD) are the major cause of early mortality and morbidity in industrialized countries (1). The prevalence of classical cardiovascular (CV) risk factors (CVRFs) such as hypertension, dyslipidemia, obesity and diabetes varies widely between different countries, and shows some important secular trends (2; 3). As an example, in many industrialized but also developing countries, the incidence of obesity and Type 2 diabetes has been increasing over the last decades at a very fast rate, so that these diseases are now reaching epidemic proportions. It has been suggested that this so-called diabesity epidemic and its associated mortality may jeopardize the increased life expectancy observed during the last century.

Hypertension, obesity, dyslipidemia and diabetes mellitus have an important genetic component (4; 5). These conditions, however, are genetically complex, and only a small fraction of these diseases are accounted for by Mendelian forms. The availability of large case-control genome-wide association studies has led to the identification of susceptibility genes for common conditions, with even modest effects. Population-based studies have additional advantages; they make it possible to perform association studies for any continuous phenotypic trait which has been properly monitored, as well as for categorical traits using extreme discordant case-control designs, as long as these conditions are sufficiently prevalent. In addition, they offer the opportunity to explore the genetic determinants of complex phenotypes such as the metabolic syndrome. Finally, this type of studies provide the opportunity to perform re-sequencing analysis on extremes of the distribution, and to identify rare genetic variants with a strong phenotypic effect (6). The success of such studies relies on a large collection, detailed and standardized phenotypes, strong analytical capabilities, replication sets and extensive genotyping (7). To harness the power of these technologies, we designed the CoLaus study. The major goals of the CoLaus Core study, described here, were to get a snap picture of the CVRFs in a particular population and to elucidate the molecular architecture of isolated CVRFs, as well as clusters of CVRFs like the metabolic syndrome. In addition, sub-studies designed to assess the psychiatric characteristics of this population as well as functional CV measurements, were nested onto this study, and will be described separately. To increase the chances of success in our effort to identify the genetic variants associated with these conditions, we selected only Caucasian individuals, thus keeping the population genetically more homogenous and minimizing population stratification. Given the allele frequency of most SNPs on the Affymetrix chip and the genetic effect sizes expected for complex diseases (i.e. odds ratio 1.3 and above—we chose to include ˜6000 individuals, so as to have enough power (˜80%) to detect genetic associations for diseases with a prevalence ˜15%.

### Results

### Recruitment Process and Sample Size

The Study was approved by the Institutional Ethic's Committee of the University of Lausanne and recruitment took place in the city of Lausanne in Switzerland, a town of 117,161 inhabitants, of which 79,420 are of a Swiss nationality (http://www.lausanne.ch/view.asp?Domld=63584) data for 2003; site assessed July 2007

The complete list of the Lausanne inhabitants aged 35-75 years (n=56,694 in 2003) was provided by the population register of the city and served to sample the participants to the study. All subjects living in the city of Lausanne in 2003 for more than 90 days have their name included in this register. The register had information on age and gender but no information regarding ethnicity or country of origin. A simple, non-stratified random selection of 19,830 subjects, corresponding to 35% of the source population, was drawn using STATA software (Stata Corp, College Station, USA), and a letter inviting the addressee to participate in the study was sent to these individuals.

Subjects who volunteered to participate were contacted by phone within 14 days by one of the staff members to set up an appointment. Subjects who didn't answer were sent a second invitation letter. If no answer was obtained, they were contacted by phone. Subjects were considered as non-participants if they refused to participate and as non-responders if contact couldn't be made after two successive letters and three successive phone calls. Individuals who didn't live in Lausanne any longer, who were dead or who didn't meet the age criteria were considered as non-eligible.

### Inclusion Criteria

The following inclusion criteria were applied: a) written informed consent; b) age 35-75 years and c) Caucasian origin. Caucasian origin was defined as having both parents and grandparents born in a restricted list of countries (available from the authors). No other exclusion criteria were applied. Since ethnicity could only be established during the clinic visit, some participants were assessed but were not included in the CoLaus study. Their data will be described elsewhere.

### Assessment Process

Participants were asked to attend the outpatient clinic at the Centre Hospitalier Universitaire Vaudois (CHUV) in the morning after an overnight fast. They had to take their medication as usual. Data were collected by trained field interviewers in a single visit lasting about 60 minutes. Informed consent was obtained from participants upon their arrival at the study clinic. The first questionnaire mailed with the appointment's letter and completed by the participant prior to the morning visit was then quickly reviewed and a second questionnaire was applied by interview prior to clinical measurements and blood collection.

### Questionnaire Data

The first set of questionnaires recorded information on demographic data, socioeconomic and marital status, and several lifestyle factors, namely tobacco, alcohol and caffeine consumption, physical activity and mood. Data on smoking included the previous and current smoking status as well as the amount of tobacco smoked (number of cigarettes, cigarillos, cigars or pipes), age of beginning and end (for former smokers). Similarly, data on alcohol consumption included the past and current drinking status as well as the number of alcoholic beverage units (wine, beer and spirits) consumed over the week preceding the interview. Caffeine consumption was assessed by the number of caffeine-containing beverages consumed per day. Personal history of overweight and/or obesity and birth weight were also collected. Finally, the 12-item General Health Questionnaire (GHQ12) (8) was applied in order to screen for the presence of non-psychotic psychiatric disorders.

The second questionnaire, administered during a face-to-face meeting with the recruiter, focused on personal and family history of disease and CV risk factors. Subjects were asked which disease(s) they or their family had presented. When a positive answer was given, further information regarding age of occurrence and number of family members affected was collected. When appropriate, death of parents was recorded with age and cause of death. Regarding blood pressure (BP) status, subjects indicated if they had been diagnosed with hypertension and subsequently if they had been, or were being treated currently for this condition. BP levels before the beginning of treatment were sought for and the names of the anti-hypertensive drugs that had been prescribed were collected. In case the anti-hypertensive regimen had been modified, the duration and the reason for changing were also recorded. Personal medicines, including prescription and self-prescribed drugs, vitamin and mineral supplements, homeopathy or natural remedies were collected, together with their main indications. In women, further data regarding reproductive and obstetrical history, oral contraception and hormonal replacement therapy was collected. Finally, an additional frailty questionnaire (for subjects aged over 50 years) and the Mini-Mental State Evaluation (MMSE—for subjects aged over 65 years) were further administered (9).

### Clinical Data

Body weight and height were measured with participants standing without shoes in light indoor clothes. Body weight was measured in kilograms to the nearest 0.1 kg using a SECA® scale (Hamburg, Germany), which was calibrated regularly. Height was measured to the nearest 5 mm using a SECA® height gauge (Hamburg, Germany). Body mass index (BMI) was defined as weight/height². Obesity was defined as BMI≧30 kg/m² and overweight as BMI≧25 kg/m² and <30 kg/m².

BP and heart rate were measured thrice on the left arm, with an appropriately sized cuff, after at least 10 minute rest in the seated position using an OMRON® HEM-907 automated oscillometric sphygmomanometer (Matsusaka, Japan) (10). The average of the last two measurements was used for analyses. Hypertension was defined as a systolic BP (SBP)≧140 mm Hg and/or a diastolic BP (DBP)≧90 mm Hg during the visit and/or presence of anti-hypertensive drug treatment and was considered as known if the subject was aware of this condition.

In addition, waist and hip circumferences were measured as recommended (11) and fat and fat-free mass were assessed by electrical bioimpedance (12) using the BODYSTAT® 1500 analyser (Isle of Man, British Isles). Finally, baldness and its age of onset were assessed in men using the Hamilton scale (13).

### Biological Data

Venous blood samples (50 ml) were drawn after an overnight fast, and most clinical chemistry assays were performed by the CHUV Clinical Laboratory on fresh blood samples whereas Pathway Diagnostics (Los Angeles, Calif.) measured adiponectin, leptin and insulin. Additional aliquots were stored at −80° C. Information on analytical procedures is available in Appendix: Supplementary Table 1.

LDL-cholesterol was calculated with the Friedewald formula only if triglycerides <4.6 mmol/l. Low HDL cholesterol level was defined as <1.0 mmol/L; high HDL cholesterol as ≧1.6 mmol/L; high LDL cholesterol was defined as ≧4.1 mmol/L and high triglyceride level was defined as ≧2.2 mmol/L (14). In our analysis, dyslipidemia was defined as low HDL cholesterol and/or high triglyceride and/or LDL cholesterol ≧4.1 mmol/L or ≧2.6 mmol/L in presence of self-reported history of myocardial infarction, stroke, coronary artery disease or diabetes.

Diabetes was defined as fasting plasma glucose ≧7.0 mmol/L and/or presence of oral hypoglycaemic or insulin treatment. Type 2 diabetes mellitus (T2DM) was defined in case of diabetes without self-reported Type 1 DM. Diabetes was considered as known if the subject was aware of this condition. Impaired fasting glucose (IFG) was defined as fasting plasma glucose between 6.1 and 6.9 mmol/L without anti-diabetic treatment (15). Furthermore, a random subgroup of the CoLaus study participants also performed an oral glucose tolerance test using the standard protocol. Results from this subgroup will be reported separately.

A urine sample was collected for the assessment of creatinine and albumin and the albumin-to-creatinine ratio was calculated. Microalbuminuria was defined as a value of albumin-to-creatinine ratio above 30 mg/g.

### Genotyping and Quality Controls

Nuclear DNA was extracted from whole blood for whole genome scan analysis. Subjects consented for the genetic data to be used for the study of cardiovascular risk factors, and associated diseases including mood disorders. Genotyping was performed on 6015 participants' samples, using the Affimetrix 500K SNP chip, as recommended by the manufacturer. A total of 379 samples were showing efficiency <85%, so that genetic data from 5636 individuals are available for this analysis.

### Data Management, Security and Quality Control

Data were entered into a secured, internet-based database. The database was designed to confirm the validity of the identification codes, establish the completeness of the information keyed in and to perform basic data checks. All discrepancies were recorded in the case report form kept in a locked room. Each modification of the data was automatically recorded, including the identity of the investigator who made the modification, the date, the old and the new value.

Staff members were trained and certified before being involved actively in the study. Certification included ability to conduct interviews, to perform phlebotomy and to process blood samples, to accurately measure anthropometric and BP levels and to enter data into electronic databases. The accurateness of the data was checked by an external quality control organization (PRN, North Hampshire, United Kingdom).

Finally, the ‘Laboratoire Central du CHUV’ is ISO 9001 certified and is regularly checked by the “Centre Suisse de Controle de Qualité” (CSCQ—Swiss Centre for Quality Control).

### Power Estimates

Power calculations were done using the program Quanto v1.1 (James Gauderman, University of Southern California, USA) (16). To estimate power in unmatched case-control studies, an arbitrary allelic frequency of 0.3, an additive model, a disease prevalence of 50% and a type 1 error rate of 10⁻⁷, taking into account 500,000 genetics markers were used. Curves were drawn for estimated genetic effect sizes (odds ratios) of 1.2 to 1.8. To estimate power in a continuous trait analysis, SBP was used as a continuous outcome for independent subjects, with an additive model and a type 1 error rate of 10⁻⁷. Curves were drawn for various minor allele frequencies (0.1 to 0.5).

### Statistical Analysis

Statistical analyses other than genome-wide association analyses were performed using Stata 9.1 (Stata Corp, College Station, USA). Results were expressed as mean±standard deviation (SD) or as number of subjects and (percentage). Data for age group 35-75 years and for the canton de Vaud were extracted from the MONICA population surveys and used to assess trends (17). Age at sampling was used for comparisons between the initial population, the random sample and the CoLaus study population, whereas age at examination was used to describe the CoLaus Study group characteristics. Comparisons were performed using Student's t-test or chi-square test for quantitative and qualitative variables, respectively. Statistical significance was assessed for p<0.05.

Association between gene variants and binary traits was assessed by logistic regression analysis using additive models and adjustment for age, gender, alcohol use and physical activity. Analysis were carried out using PLINK version 0.99 p (http://pngu.mgh.harvard.edu/purcell/plink/) on 5 different SNPs linked to gene variants previously shown to be associated with CVRFs, namely FTO for overweight and obesity (18), LPL for dyslipidemia (19), cholinergic nicotinic receptor 3 (CHRNA3) for nicotinic dependence (20), TCF7L2 for diabetes (21), and the 9 p21 locus for coronary artery disease (22).

### Sponsoring

The Study was sponsored in part by GlaxoSmithKline and all participants were duly informed about this sponsorship and consented for the use of biological samples and data by GlaxoSmithKline and its subsidiaries.

### Results

### Recruitment

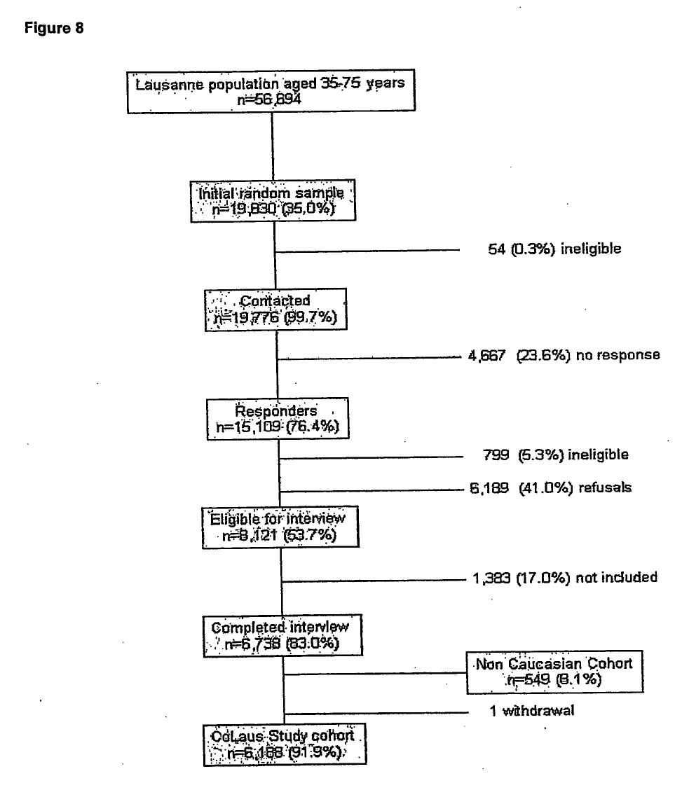

Recruitment began in June 2003 and ended in May 2006. The sampling procedure is summarized in [FIG. 8]. Of the initial 19,830 subjects sampled, 54 subjects were considered as non-eligible before contact and 15,109 (76%) responses were obtained. A total of 4667 subjects who did not respond were considered as non-responders. Among responders, 6,189 (41%) subjects refused to participate in the study and 799 (5%) were considered as non-eligible. Among the latter, 53% moved to a different city, 32% were out of the age range and 11% were reported to be deceased. The sample of 8,121 subjects who agreed to participate represented 41% of the initially sampled population, 54% of all responders and 57% of all eligible responders.

Among these subjects, the first 6,738 were invited to attend the clinic and completed the examination. 6,189 participants met the inclusion criteria (including ethnicity) and were included in the CoLaus study. The 549 participants (8.1%) were not of Caucasian ethnicity and were excluded from the CoLaus study. As the number of subjects who agreed to participate (8,121) was higher than the number of subjects initially planned for the CoLaus study (6,000), 1,383 could not be included into the study although they were willing to participate. One subject withdrew after consent due to personal reasons. Therefore, the final CoLaus sample (n=6,188) represents 43% of the eligible responders, 41% of all the responders and 31% of the initially sampled population.

The gender and age characteristics of the source population, the initial random sample and the final CoLaus sample are summarized in [Appendix Table I]. Overall, both the sampled population and the CoLaus study participants were on average one year younger than the base population, due to an under-representation of subjects aged over 65 years while no differences were found for gender distribution. Further, after excluding non-eligible subjects, no differences were found regarding mean age and gender distribution between the subjects included and the random sample (not shown). The CoLaus Study participants were significantly older (51.1±0.1 vs. 50.8±0.1 years, p<0.005) than the random sample, while no differences were found for gender distribution (not shown). Age at examination of the participants was on average 2 years higher than age at sampling because of the time elapsed between the two procedures. Distribution of the zip codes within the city was comparable between the base population, the random sample and the CoLaus study participants (not shown).

### Socio-Economic Characteristics of the CoLaus Study Participants

The CoLaus Study participants' main characteristics are summarized in Appendix Table II. No gender differences were found regarding the percentage of foreigners. Overall, women were more frequently divorced, widowed or single than men and thus were living more often alone or as a monoparental family (41.7% vs. 23.6%, p<0.01). Women were also less frequently on a full-time job, more prone to receive social help (26.2% vs. 22.2%, p<0.001) and had a lower educational level. Among subjects aged less than 40 years old, 48.4% of women had a high school/college/university degree versus 47.4% of men (p<0.01). Clinical characteristics of the CoLaus Study participants

Men had a higher BMI, waist/hip ratio, systolic and DBP levels, but lower body fat percentage, than women. (Appendix: Tables IIIa and IIIb). Men had higher total cholesterol and triglyceride levels, but lower HDL cholesterol, than women. Fasting blood glucose levels, homocystein, apolipoprotein B, insulin, NT-proBNP, were higher in men; conversely, women presented higher LDL cholesterol particle size, adiponectin, leptin and hsCRP than men.

### CVRFs Within the CoLaus Study

More than one third of the overall sample was overweight, and slightly less than one-sixth was obese; overweight and obesity were also more prevalent in men (Appendix Table IV) and increased with age. In men aged 35-44 years, the prevalence of overweight and obesity were 40.7% and 11.4%, respectively, whereas in men aged 65-75 the corresponding figures were 50.8% and 22.7% (p<0.001). The corresponding figures for women were 21.9% and 9.6%, and 35.8% and 17.5% (p<0.001), respectively.

Smoking was reported by 27% of the participants; the prevalence of current smoking was higher in men and tended to decrease with age, from 35.3% among 35-44 year olds to 20.7% among 65-75 year olds in men (p<0.001), the corresponding figures being 28.1% and 14.7% in women (p<0.001). Interestingly, in the 45-54 age class, prevalence of smoking was higher in women than in men (30.7% vs. 28.8% (p<0.001)).

The prevalence of hypertension was 36.7% overall, was higher in men and increased with age: 18.3% and 75.1% in men aged 35-44 and 65-75, respectively (p<0.001); the corresponding numbers in women were 9.9% and 59.1% (p<0.001). Among hypertensive subjects, 50.1% were currently taking anti-hypertensive medication. Treatment for hypertension was more frequent in female subjects and increased with age (from 32.2% to 59.4%). Of the treated hypertensive subjects more than half (52.0%) had BP levels ≧140/90 mmHg.

High LDL cholesterol, high triglyceride and low HDL cholesterol levels were seen in 20.8%, 12.5% and 2.8% of the subjects, respectively and these conditions were also more prevalent in men. The prevalence of high HDL cholesterol was 53.4% and higher in women than men (71.6% vs 33.2%). Overall, one third of the sample had dyslipidemia, the prevalence of which was higher in men (42.7%) than in women (26.6%).

Use of statin therapy in subjects with high LDL cholesterol was 5.6%. Prevalence of statin therapy was 69% in subjects after myocardial infarction, 33% in subjects after stroke and 70% in subjects after coronary artery bypass graft. Target level of LDL-cholesterol for secondary prevention, as recommended (<2.6 mmol/l), was achieved in 28%, 38%, 28% and 33% of the subjects with diabetes, myocardial infarction, stroke and coronary artery bypass graft, respectively.

The overall prevalence of diabetes was 6.6%, and was higher in men. Nine subjects (2.2%) reported to have TIDM. The prevalence of diabetes increased with age, from 2.5% to 17.2% in men aged 35-44 and 65-75, respectively; the corresponding numbers were 1.2% and 9.0% in women with a peak prevalence of 17.2% in men aged>65. Roughly a third were newly diagnosed diabetics (31.5% for women and 34.7% for men, respectively). The prevalence of IFG in the CoLaus population was 9.8% and was higher in men than in women (14.3% vs 5.6%).

Treatment for diabetes was present in 73.9% of diabetic subjects but nearly all known diabetics were treated (96.3%). Of the treated diabetic subjects 63.9% had a fasting blood glucose ≧7 mmol/L, at the time of their visit.

Finally, 0.7% of women and 2.6% of men reported a personal history of myocardial infarction (p<0.001); conversely, no gender differences were found regarding personal history of stroke: 1.0% of women vs. 1.3% of men, p=0.16.

### Power Estimates

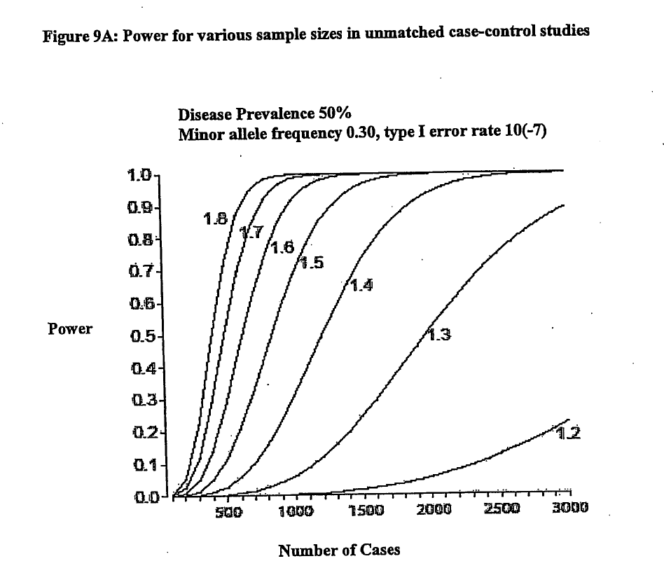

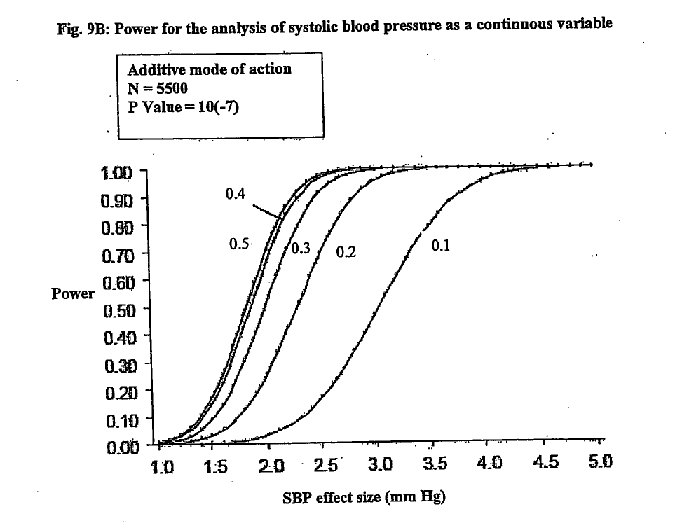

When using an unmatched case-control study design, the CoLaus study may have significant power to study genotype/phenotype associations, depending on the number of cases and estimated effect size [FIG. 9A]. For hypertension with 2268 cases, the estimated power is 0.9 for a 1.4 effect size and 0.5 for a 1.3 effect size. Cases for main CVRFs in the CoLaus study are: dyslipidemia: 2021, obesity: 974, smoking (>25 cigarettes/day): 746, Type 2 diabetes: 398, coronary heart disease: 262, low HDL: 170. For continuous trait analysis, the example of SBP was taken [FIG. 9B]. For allelic frequencies of 0.2 to 0.4, the study has an estimated power of >0.8 to detect BP variations of 2.0-2.3 mm Hg.

Association of Selected Gene Variants with Cardio-Vascular Related Traits

Selected gene variants known to be associated with CVRFs or CV disease were used as positive controls for initial genetic analyses. In binary trait association studies, we found a significant effect of the FTO gene on overweight/obesity with an OR of 1.3 (CI 1.1-1.5) (p<0.005) (Appendix: Table V). Additional significant associations were found for LPL and dyslipidemia: OR 0.6 (CI 0.4-0.7) (p<0.005), CHRNA3 and nicotinic dependence: OR 0.8 (CI 0.7-0.9) (p<0.005), the 9 p21 locus and coronary artery disease: OR 1.8 (CI 1.2-2.8) (p=0.02), and the TCF7L2 gene with diabetes: OR 1.6 (CI 1.3-2.0) (p<0.005).

### Discussion

Described in this report are the rationale, objectives, methods and first results from the CoLaus study, a single-center population-based sample including 6188 extensively phenotyped Caucasian subjects aged 35-75. Our results indicate that the prevalence of major CVRFs is high, in particular in men, and that this collection represents a powerful tool to replicate known genotype/phenotype associations and, most importantly, to identify new molecular determinants of CVRFs and associated diseases.

The participation rate of 41% in the CoLaus study is comparable to the MONICA surveys conducted in Switzerland and in France (23). The lower response rate among elderly subjects is in agreement with previous data (24) and might be related to a lower interest for the study. However, the distribution of age groups 35-54 and 55-75 in the CoLaus study was comparable to the source population (Appendix Table I). Also, there was no gender or zip code distribution difference between the source population, the random sample and the CoLaus participants. Although the optimal sampling frame would have consisted of a list of all Caucasians living in the city, information on ethnicity was not available to the investigators before examination and it is not possible to assess whether ethnicity had an effect on the participation rate.

The prevalence of main CVRFs was high in the CoLaus participants. Roughly over half of the participants presented with overweight and obesity, over a third had high BP or dyslipidemia while one in 15 participants had diabetes.

In agreement with the literature (25), men had a higher prevalence of obesity and overweight than women. Further, comparison with data from the MONICA study suggests that the prevalence of obesity is increasing, which confirms what has been found in the nearby city of Geneva (26). The higher prevalence of overweight in men might also account for their higher prevalence of diabetes and hypertension. In clinical practice, the diagnosis of hypertension relies on several consecutive BP measurements but as in most epidemiological studies, BP measurements were conducted during a single visit (27). Terminal digit preference in BP readings may induce a bias, a consistency check was conducted showing no significant deviation of terminal digit frequency from the 10% value for all BP measurements (not shown). The prevalence of hypertension was higher in men than in women and increased with age. Interestingly, the gender-specific prevalence rates found in the CoLaus study were similar to those previously reported for the same age group in Italy (28) and in nearby Geneva (26). We also observed that over half of the treated hypertensive subjects had a BP≧140/90 mmHg at the time of their examination, indicating the continuous need to improve treatment compliance. The prevalence of diabetes was 6.6% and was significantly higher in men and in particular after the age of 55 years. These numbers are similar to other Swiss estimates (29) and to the KORA Augsburg study in the southern part of Germany (30). About a quarter of the CoLaus participants were smokers, a figure quite similar to the data reported for the Geneva population in 2003 (26). The prevalence of smoking was higher in men than in women, although this difference tended to decrease among younger age groups, as previously reported (31). Indeed, in the age group 45-54, the prevalence of smoking was higher in women than in men, suggesting that the “gender gap” regarding smoking no longer exists for middle-aged subjects.

The high prevalence of CVRFs in this study population underscores the necessity to increase disease awareness, to improve screening in high risk subjects and to promote prevention both at the public health and individual level.

Recently, an increasing number of reports have demonstrated the power of whole genome scan association studies approach in complex diseases such as diabetes (32) and obesity (18). The size of the CoLaus study, the population-based design and the in-depth phenotypisation were chosen in order to harness the power of this technology using dichotomous and continuous trait analyses. In particular, this ensures an identical phenotypisation in cases and controls, which has been a limitation in some of the recently published reports (33). We were able to replicate known genotype/phenotype associations for major CVRFs. In particular, we confirmed the association of several variants within the FTO gene with obesity, waist circumference and percent fat mass. We also observed an association of the lipoprotein lipase gene with atherogenic dyslipidemia, the 9 p21 region with coronary artery disease and CHRNA3 with nicotinic dependence. Additional analyses are currently ongoing.

### Perspectives

A more comprehensive characterization of the Colaus participants is currently ongoing. First all participants aged 35 to 65 are solicited to undergo a psychiatric investigation based on a semi-structured diagnostic interview and in 500 subjects we assessed CV functional measurements. Indeed several previous studies have revealed associations between mood disorders and, in particular depression, and CVRFs and CV diseases (reviewed in (34)). The availability of a simultaneous CV and psychiatric phenotype will allow us to further explore the epidemiologic and potentially a genetic basis for this association.

Finally, a longitudinal follow-up of all participants in the CoLaus study is planned and shall provide essential data for trends over time of major CVRFs and on incident cases of CV diseases. In the current cross-sectional study, participants were asked whether they consented to be contacted for follow-up, with and over 90% favorable responses.

### CONCLUSIONS

In summary, these initial results from the CoLaus study show that the prevalence of main CVRFs is high in the Caucasian population of Lausanne, and in particular in men. This emphasizes the need for continued epidemiological monitoring and for strengthening interventions to reduce the prevalence and severity of CVRFs in this population. This population-based study with over 6000 extensively characterized and genotyped participants constitutes a unique resource to replicate known, or identify new, molecular determinants of CVRFs and associated diseases.

### List of Abbreviations Used

CV: cardiovascular; CVD: cardiovascular disase; CVRF: cardiovascular risk factor; BP: blood pressure; SBP: systolic blood pressure, DBP: diastolic blood pressure; BMI: body mass index; LDL-cholesterol: low-density cholesterol; HDL-cholesterol: high density cholesterol; NT-proBNP: N-terminal pro-Brain Natriuretic Peptide; ASAT or AST: aspartate aminotrasnferase; ALAT or ALT: alanine aminotransferase; Gamma-GT or GGT: gamma glutaryl transferase; CDT: carbohydrate deficient transferine; hs CRP: high sensitivity C-reactive protein. CAD: coronary artery disease, T1D/T2D: Type 1/Type 2 diabetes mellitus.

### Competing Interests: None Declared.

Kijoung S. Song, Xin Yuan, Theodore M. Danoff, Heide A. Stimadel, Dawn Waterworth and Vincent Mooser are full-time employees of GlaxoSmithkline.

### Authors' Contributions:

Design and conception of the CoLaus study: Vollenweider, Waeber, Mooser, Waterworth, Stimadel, Danoff, Paccaud, Hayoz, Presig. Coordination of the CoLaus project: Vollenweider; Mooser, Waeber, Pécoud, Hayoz, Waterworth. Providion of participants and study material: Firmann, Mayor, Vollenweider, Waeber, Pécoud, Hayoz, Paccaud. Collection and assembly of the data: Firmann, Mayor, Marques Vidal, Bochud, Song, Yuan. Statistical expertise: Marques Vidal, Firmann, Bochud, Song, Yuan. Analysis and interpretation of the data: Vollenweider, Firmann, Weaber, Mooser, Waterworth, Strinadel, Marques Vidal. Drafting of the article: Vollenweider, Firmann, Marques Vidal. Critical revision of the article: Vollenweider, Firmann, Mayor, Mooser, Waeber, Marques Vidal, Bochud, Preisig. Final revision of the article: Vollenweider, Mooser, Firmann, Waeber, Marques Vidal, Bochud, Paccaud, Hayoz, Waterworth, Stimadel, Danoff.

### Acknowledgements

The CoLaus study was supported by research grants from GlaxoSmithKline and from the Faculty of Biology and Medicine of Lausanne, Switzerland.

The authors would like to express their gratitude to the participants in the Lausanne CoLaus study, to the investigators who have contributed to the recruitment, in particular Yolande Barreau, Anne-Lise Bastian, Binasa Ramic, Martine Moranville, Martine Baumer, Marcy Sagette, Jeanne Ecoffey and Sylvie Mermoud for data collection, and to Allen Roses, and Lefkos T. Middleton for their support.

### REFERENCES

(1) Mathers et al., Plos Med 2006; 3(111)e442.
(2) Kearney et al., The Lancet 2005; 365(9455):217-223
(3) King et al., Diabetes Care 1998; 21(9):1414-1431
(4) Caulfield et al. The Lancet 2003; 361(9375):2118-2123
(5) Rankinen et al., Obesity 2006; 14(4):529-644
(6) Romeo et al., Nat Genet 2007; 39(4):513-516
(7) Chanock et al., Nature 2007; 447(7145):655-660.
(8) Worsley et al., The Australian and New Zealand J. of Psychiatry 1977; 11(4):260-272
(9) Folstein et al., J Psychiatr Res 1975; 12(3): 189-198
(10) El Assaad et al. Blood Press Monitoring 2002; 7(4):237-241.
(11) Lean et al. Brit Med J 1995; 311(6998):158-161.
(12) Jebb et al. Int J Obesity (Lond) 2007; 31(5):756-762
(13) Hamilton J B. Ann N Y Acad Sci 1951; 53(3):708-728.
(14) Third Report of the National Cholesterol Education Program (NCEP) Expert Panel on Detection, Evaluation, and Treatment of High Blood Cholesterol in Adults (Adult Treatment Panel III) final report. Circulation 2002; 106(25):3143-3421.
(15) Alberti et al., Diabet Med 1998; 15(7):539-553
(16) Gauderman W J and Morrison J M. QUANTO 1.1. A computer program for power and sample size calculations for genetic-epidemiologic studies. http://hydra.usc.edu/gxe. 2006.
(17) Wietlisbach et al., Prev Med 1997; 26(4):523-533
(18) Frayling et al., Science 2007; 316(5826):889-894.
(19) Hu et al., J Lipid Res 2006; 47(9):1908-1914.
(20) Saccone et al., Hum Mol Genet 2007; 16(1):36-49.
(21) Grant et al. Nature Genetics 2006; 38(3):320-323.
(22) Helgadottir et al., Science 2007; 316 (5830):1491-1493.
(23) Wolf et al., Participation rates, quality of sampling frames and sampling fractions in the MONICA surveys. Sep. 15, 1998. Helsinki, Finland, WHO MONICA. Quality assessment reports. Ref Type: Report
(24) Groves and Couper, Nonresponse in household interview surveys. New York: John Wiley & Sons, Inc.; 1998.
(25) Schokker et al., Obes Rev 2007; 8(2):101-107
(26) Galobardes et al., Ann Epidemiol 2003; 13(7):537-540.
(27) Wolf-Maier et al., JAMA 2003; 289(18):2363-2369.
(28) Giampaoli et al., Ital Heart J Suppl 2001; 2(3):294-302
(29) Nedeltchev et al., Gesundheitswesen 2005; 67 Suppl 1: S115-S121
(30) Herder et al., Prev Med 2006; 42(5):348-353
(31) Chiolero et al., Prev Med 2006; 42(5):348-353.
(32) Scott et al., Science 2007; 316(5829):1341-1345
(33) Genome-wide association study of 14,000 cases of seven common diseases and 3,000 shared controls. Nature 2007; 447(7145):661-678.
(34) Musselman et al., Arch Gen Psychiatry 1998; 55(7):580-592.

APPENDIX TABLE I
Distribution of participants by age and sex in the source population,
the initial random sample and the CoLaus Study participants.

| | Source population | Random sample | | CoLaus Study | |
|---|---|---|---|---|---|
| | (n = 56,694) | (n = 19,830) | | (n = 6,188) | |
| Women (%) | 30,141 (53.4) | 10,601 | (53.5) | 3,251 | (52.5) |
| P value | | 0.79 | | 0.08 | |
| Age (years) | 52.0 ± 11.6 | 50.8 ± 11.5 | | 51.1 ± 10.9 | |
| P value | | <0.001 | | <0.001 | |
| Age group | | | | | |
| 35-44 | 18.877 (33.4) | 7,265 | (36.6) | 2,051 | (33.1) |
| 45-54 | 14,614 (25.9) | 5,202 | (26.2) | 1,682 | (27.2) |
| 55-64 | 12.484 (22.1) | 4,417 | (22.3) | 1,657 | (26.8) |
| 65-75 | 10,524 (18.6) | 2,946 | (14.9) | 798 | (12.9) |

| | | |
|---|---|---|
| P value | <0.001 | <0.001 |

Results are expressed as number of subjects and (percentage) or as mean ± SD. Statistical analysis by Student's t-test or chi-square test compared to the source population

APPENDIX TABLE II
Socio-economic characteristics of
participants in the CoLaus Study.

| | Overall | Women | | Men | | P |
|---|---|---|---|---|---|---|
| | (n = 6,188) | (n = 3,251) | | (n = 2,937) | | value |
| Born in | 3,997 (64.6) | 2,129 | (65.5) | 1,868 | (63.6) | 0.12 |
| Switzer- | | | | | | |
| land (%) | | | | | | |
| Marital | | | | | | |
| status (%) | | | | | | |
| Single | 1,022 (16.5) | 569 | (17.5) | 453 | (15.4) | |
| Married | 3,635 (58.8) | 1,673 | (51.5) | 1,962 | (66.9) | <0.001 |
| Divorced | 1,242 (20.1) | 761 | (23.4) | 481 | (16.4) | |
| Widowed | 287 (4.6) | 248 | (7.6) | 39 | (1.3) | |
| Education | | | | | | |
| (%) | | | | | | |
| Basic | 1,287 (20.8) | 777 | (23.9) | 510 | (17.4) | |
| Apprentice- | 2,286 (37.0) | 1,170 | (36.0) | 1,116 | (38.0) | <0.001 |
| ship | | | | | | |
| High | 1,470 (23.9) | 804 | (24.8) | 666 | (22.7) | |
| school/ | | | | | | |
| college | | | | | | |
| University | 1,140 (18.4) | 497 | (15.3) | 643 | (21.9) | |
| Work | | | | | | |
| status (%) | | | | | | |
| Full time | 3,790 (61.2) | 1,694 | (52.1) | 2,096 | (71.4) | <0.001 |
| Other | 2,398 (38.8) | 1.557 | (47.9) | 841 | (28.6) | |

Results are expressed as number of subjects and (percentage). Comparison between gender was performed using chi-square test.

APPENDIX: TABLE IIIa
Clinical characteristics of participants in the CoLaus study, by gender.

| | Overall | Women | Men | P |
|---|---|---|---|---|
| | (n = 6,188) | (n = 3,251) | (n = 2,937) | value |
| Age (years) | 53.1 ± 10.8 | 53.5 ± 10.7 | 52.6 ± 10.8 | <0.001 |
| Waist/hip ratio | 0.88 ± 0.08 | 0.83 ± 0.07 | 0.93 ± 0.06 | <0.001 |
| BMI (kg/m²) | 25.8 ± 4.6 | 25.1 ± 4.9 | 26.6 ± 4.0 | <0.001 |
| Body fat (%) | 29.3 ± 9.0 | 34.4 ± 8.2 | 23.8 ± 6.1 | <0.001 |
| Systolic BP (mm Hg) | 128 ± 18 | 125 ± 18 | 132 ± 17 | <0.001 |
| Diastolic BP (mm Hg) | 79 ± 11 | 78 ± 11 | 81 ± 11 | <0.001 |
| Total cholesterol (mmol/L) | 5.59 ± 1.04 | 5.61 ± 1.03 | 5.56 ± 1.04 | <0.05 |
| HDL cholesterol mmol/L) | 1.63 ± 0.44 | 1.81 ± 0.43 | 1.44 ± 0.36 | <0.001 |
| Triglycerides (mmol/L) | 1.40 ± 1.18 | 1.16 ± 0.66 | 1.66 ± 1.52 | <0.001 |
| LDL cholesterol particle size (nm) | 271 ± 4 | 273 ± 4 | 271 ± 5 | <0.001 |
| Apolipoprotein B (mg/dL) | 1.74 ± 1.34 | 1.69 ± 1.29 | 1.80 ± 1.38 | <0.005 |
| Glucose (mmol/L) | 5.55 ± 1.15 | 5.34 ± 1.02 | 5.78 ± 1.23 | <0.001 |
| Insulin (□U/mL) | 8.44 ± 6.3 | 7.97 ± 5.47 | 9.62 ± 6.78 | <0.001 |
| Adiponectin (□g/mL) | 9.94 ± 8.12 | 12.32 ± 9.33 | 7.32 ± 5.43 | <0.001 |
| Leptin (ng/mL) | 13.1 ± 10.7 | 16.9 ± 11.7 | 8.65 ± 7.3 | <0.001 |
| Homocystein (□mol/L) | 10.4 ± 4.4 | 9.4 ± 3.2 | 11.4 ± 5.2 | <0.001 |
| hsCRP (mg/L) | 2.49 ± 3.48 | 2.65 ± 3.71 | 2.30 ± 3.21 | <0.001 |
| Pro-BNP (ng/L) | 682 ± 531 | 679 ± 519 | 686 ± 545 | 0.60 |

Results are expressed as mean ± SD. BMI: body mass index; HDL: high density lipoprotein, hsCRP: high sensitivity C-reactive protein, BNP: brain natriuretic peptide. Statistical analysis between gender by Student's t-test or chi-square test.

APPENDIX TABLE IIIb
Clinical characteristics of participants of CoLaus study, by gender.

| | Overall | Women | Men | P |
|---|---|---|---|---|
| | (n = 6,188) | (n = 3,251) | (n = 2,937) | value |
| ASAT (U/L) | 29.96 ± 14.04 | 26.22 ± 9.88 | 34.10 ± 16.57 | <0.001 |
| ALAT (U/L) | 27.84 ± 19.50 | 21.98 ± 14.47 | 34.32 ± 22.13 | <0.001 |
| Alkaline phosphatase (U/L) | 63.47 ± 20.72 | 62.66 ± 20.99 | 64.38 ± 20.38 | <0.001 |
| Gamma- GT (U/L) | 33.20 ± 59.04 | 22.95 ± 25.78 | 44.57 ± 79.80 | <0.001 |
| Calcium (mmol/L) | 2.29 ± 0.09 | 2.28 ± 0.10 | 2.29 ± 0.09 | <0.001 |
| Albumin (g/L) | 44.20 ± 2.53 | 43.73 ± 2.48 | 44.71 ± 2.48 | <0.001 |
| Total protein (g/L) | 74.41 ± 4.39 | 73.93 ± 4.42 | 74.95 ± 4.30 | <0.001 |
| Uric acid (□mol/L) | 313.49 ± 84.47 | 270.56 ± 67.23 | 361.08 ± 75.69 | <0.001 |
| CDT (% of total transferrin) | 0.95 ± 0.81 | 0.80 ± 0.48 | 1.12 ± 1.02 | <0.001 |

Results are expressed as mean ± SD. ASAT: Aspartate amino-transferase. ALAT: Alanine amino-transferase. Gamma-GT: Gamma- glutaryl-transferase. CDT: Carbohydrate deficient transferrin. Statistical analysis between genders by Student's t-test or chi-square test.

APPENDIX TABLE IV
Prevalence of selected cardiovascular risk factors in CoLaus study participants.

| | Overall | | Women | | Men | | P |
|---|---|---|---|---|---|---|---|
| | (n = 6,188) | | (n = 3,251) | | (n = 2,937) | | value |
| BMI status (%) | | | | | | | |
| Overweight | 2265 | (36.6) | 922 | (28.4) | 1343 | (45.7) | <0.001 |
| Obesity | 974 | (15.7) | 472 | (14.5) | 502 | (17.1) | |
| Smoking status (%) | | | | | | | |
| Current | 1673 | (27.0) | 813 | (25.0) | 860 | (29.3) | |
| Former | 2034 | (32.9) | 904 | (27.8) | 1130 | (38.5) | <0.001 |
| Never | 2479 | (40.1) | 1534 | (47.2) | 945 | (32.2) | |
| Blood pressure status (%) | | | | | | | |
| Hypertension | 2268 | (36.7) | 1004 | (30.9) | 1264 | (43.0) | <0.001 |
| Treated hypertension | 1131 | (50.1) | 537 | (53.8) | 594 | (47.1) | <0.005 |
| Treated to goal | 542 | (48.0) | 271 | (50.6) | 271 | (45.6) | NS |
| Lipid status (%) | | | | | | | |
| High LDL cholesterol | 1263 | (20.8) | 631 | (19.5) | 632 | (22.2) | <0.001 |
| High triglycerides | 773 | (12.5) | 216 | (6.7) | 557 | (19.0) | <0.001 |
| Low HDL cholesterol | 170 | (2.8) | 35 | (1.1) | 135 | (4.6) | <0.001 |
| High HDL cholesterol | 3296 | (53.4) | 2324 | (71.6) | 972 | (33.2) | <0.001 |
| Dyslipidemia | 2113 | (34.2) | 862 | (26.6) | 1251 | (42.7) | <0.001 |
| Treated dyslipidemia | 286 | (13.5) | 95 | (11.0) | 191 | (15.3) | <0.001 |
| Glycaemic status (%) | | | | | | | |
| Diabetes | 407 | (6.6) | 130 | (4.0) | 277 | (9.5) | <0.001 |
| Known Diabetes | 270 | (66.3) | 89 | (68.5) | 181 | (65.3) | <0.005 |
| Treated Diabetes | 260 | (96.3) | 85 | (95.5) | 175 | (96.7) | <0.01 |
| Microalbuminuria (%) | 380 | (6.3) | 173 | (5.4) | 207 | (7.3) | |

Overweight: BMI ≧ 25 kg/m². Obesity: BMI ≧ 30 kg/m². Hypertension: Blood pressure ≧ 140/90 mmHg and/or presence of anti-hypertensive drug treatment. Low HDL cholesterol: <1 mmol/L; high HDL cholesterol: ≧1.6 mmol/L; high LDL cholesterol: ≧4.1 mmol/L and high triglyceride: ≧2.2 mmol/L. Dyslipidemia: low HDL cholesterol and/or high triglyceride and/or LDL cholesterol ≧ 4.1 mmol/L or ≧ 2.6 mmol/L in presence of self-reported myocardial infarction, stroke, coronary artery disease or diabetes. Diabetes: fasting plasma glucose ≧ 7 mmol/L and/or presence of oral hypoglycaemic or insulin treatment.

APPENDIX TABLE V
Selected candidate-gene analyses, used as positive
controls in the participants of the CoLaus study.

| CV related trait | Gene | SNP Number | Cases/Controls | OR | P Value |
|---|---|---|---|---|---|
| Overweight and | FTO | rs9939609 | 2949/2685 | 1.3 (CI 1.1-1.5) | <0.005 |
| obesity¹ | | | | | |
| Dyslipidemia² | LPL | rs17411126 | 633/678 | 0.6 (CI 0.4-0.7) | <0.005 |
| Nicotinic | CHRNA3 | rs6495308 | 665/2275 | 0.8 (CI 0.7-0.9) | <0.005 |
| dependence³ | | | | | |
| CAD⁴ | 9p21 | rs4977574 | 262/1580 | 1.8 (CI 1.2-2.8) | 0.02 |
| Diabetes⁵ | TCF7L2 | rs7901695 | 407/1221 | 1.6 (CI 1.3-2.0) | <0.005 |

Definitions for the candidate-gene analyses
¹Overweight and obesity: Cases: BMI ≧ 25 kg/m². Controls: BMI < 25 kg/m^{2.}
²Dyslipidemia: Population-specific percentiles were used. Cases: HDL-Chol < 25% ile and TG > 75% ile. Controls: HDL-Chol > 50% ile and TG < 50% ile.
³Nicotinic dependence: Cases: subjects with >25 cigarettes/day. Controls: Never smokers and 0 cigarettes/day.
⁴CAD (coronary artery disease): Cases: History of myocardial infarction and/or Coronary artery bypass grafting (CABG) and/or angina. Controls: Matched subjects by age (+/−2 years) and sex in a ratio of 1:6.
⁵Diabetes: Cases: fasting blood glucose ≧ 7.0 mmol/l and/or taking oral antidiabetic drugs or on insulin and/or history of diabetes Controls: fasting blood glucose < 6 mmol/l and not taking anti diabetic medications or insulin.

APPENDIX SUPPLEMENTARY TABLE 1
Clinical chemistry and biological makers measured in the CoLaus study with analytical
procedures, maximum inter and intra-batch coefficient of variation and manufacturer

| | | Max. inter and | |
|---|---|---|---|
| Marker | Type of assay | intra-batch CVs | Manufacturer |
| Adiponectin | ELISA | 8.3%-8.3% | R&D Systems, Inc, |
| | | | Minneapolis, USA |
| Albumin | Bromocresol green | 2.5%-0.4% | Roche Diagnostics, CH |
| Alanine | International Federation of Clinical | 5.6%-0.7% | Roche Diagnostics, CH |
| aminotransferase | Chemistry (IFCC) method at 37° C. | | |
| (ALT) | (IFCC at 37° C.) | | |
| Alkaline | (IFCC at 37° C.) | 3.3%-0.7% | Roche Diagnostics, CH |
| phosphatase | | | |
| Aspartate | (IFCC at 37° C.) | 3.6%-1.8% | Roche Diagnostics, CH |
| aminotransferase | | | |
| (AST) | | | |
| Apolipopoprotein | Turbidimetry | 8.7%-7.6% | Polymedco, Chicago, |
| B (ApoB) | | | USA |
| Calcium | O-cresolphtalein | 2.1%-1.5% | Roche Diagnostics, CH |
| Carbohydrate | separation by capillary | Maximum intra- | Beckman Coulter |
| deficient | electrophoresis (Beckman P/ACE | batch CV was | Instruments, Switzerland |
| transferin (CDT) | 5510 System) using the Ceofix- | between 3.8% | Analis, Belgium, |
| | CDT reagent kits (kits #10-004760) | (highest CDT | |
| | | levels) and 15.3% | |
| | | (lowest CDT) | |
| Cholesterol | CHOD-PAP | 1.6%-1.7% | Roche Diagnostics, CH |
| (Total) | | | |
| Creatinine | Jaffe kinetic compensated method | 2.9%-0.7% | Roche Diagnostics, CH |
| (serum and urine) | | | |
| Gamma glutamyl | Optimized standard IFCC method at | 1.6%-0.4% | Roche Diagnostics, CH |
| transferase | 37° C. | | |
| (GGT) | | | |
| Glucose | Glucose dehydrogenase | 2.1%-1.0% | Roche Diagnostics, CH |
| HDL-cholesterol | CHOD-PAP + PEG + cyclodextrin | 3.6%-0.9% | Roche Diagnostics, CH |
| High sensitive | Immunoassay and latex HS | 4.6%-1.3% | Roche Diagnostics, CH |
| CRP (hsCRP) | | | |
| Homocystein | High pressure liquid | 3.1%-2.9% | Agilent 1100 apparatus |
| | chromatography following | | |
| | ammonium 7-fluorobenzo-2-oxa-1, | | |
| | 3-diazole-4-sulphonate (SBD-F) | | |
| | derivatisation | | |
| Insulin | Solid-phase, two-site | Maximum intra- | Diagnostic Products |
| | chemiluminescent immunometric | assay CV of 13.7%. | Corporation, Los |
| | assay | | Angeles, USA |
| LDL particle size | Polyacrylamide gel electrophoresis, | 1.5%-0.5% | Quantimetrix |
| | Lipoprint LDL kit ®, | | Corporation, CA, USA |
| Leptin | ELISA | 12.8%-5.8% | American Lab. Products |
| | | | Co. Windham, USA |
| NT-proBNP | ELISA | | American Lab Products |
| | | | Co. |
| Total protein | Biuret | 1.3%-0.6% | Roche Diagnostics, CH |
| Triglycerides | GPO-PAP | 2.9%-1.5% | Roche Diagnostics, CH |
| Uric acid | uricase-PAP | 1.0%-0.5% | Roche Diagnostics, CH |

What is claimed is:

1. A method of identifying a subject's relative risk of developing metabolic syndrome, comprising determining the allelic genotype at the STK39/SPAK rs16855027 single nucleotide polymorphism loci, wherein an T/T genotype indicates said subject has a reduced risk of developing metabolic syndrome compared to an individual with an A/T or A/A genotype.

2. The method of claim 1 were said subject has a Body Mass Index of greater than 25 kg/m².

3. The method of claim 1 where said subject is of Caucasian ancestry.

4. A method of identifying a subject's relative risk of developing a condition selected from hypertension, Type 2 diabetes mellitus (T2D), cardiovascular disease, and dyslipidemia, the method comprising determining the allelic genotype at the STK39/SPAK rs16855027 single nucleotide polymorphism loci, wherein an T/T genotype indicates said subject has a reduced risk of developing said condition compared to an individual with an A/T or A/A genotype.

5. The method of claim 1 were said subject has a Body Mass Index of greater than 25 kg/m².

6. The method of claim 1 where said subject is of Caucasian ancestry.

7. A method of identifying, in a population of overweight subjects, subpopulations at different relative risk of developing a condition selected from hypertension, T2D, and cardiovascular disease, the method comprising determining the allelic genotype at the STK39/SPAK rs16855027 single nucleotide polymorphism loci for each subject in the population, where the subpopulation having an T/T genotype is at reduced risk of developing said condition compared to the subpopulation with the A/T and A/A genotypes.

8. A method of screening a test compound for use as an therapeutic for metabolic syndrome, comprising determining whether said compound inhibits the kinase activity of STK39/SPAK, where inhibitory activity indicates potential as a treatment for metabolic syndrome or the associated components or sequelae of metabolic syndrome.

9. A method of treating a condition selected from metabolic syndrome, hypertension, cardiovascular disease and T2D, comprising administering a therapeutic compound that inhibits the kinase activity of STK39/SPAK.
